(19) United States
(12) Patent Application Publication
(10) Pub. No.: US 2009/0131406 A1
(43) Pub. Date: May 21, 2009

(54) CROPWELLLINS AND SYNTHETIC DERIVATIVES THEREOF USED AS MEDICAMENTS

(75) Inventors: Horst-Peter Antonicek, Bergisch Gladbach (DE); Robert Velten, Langenfeld (DE); Peter Jeschke, Bergisch Gladbach (DE)

Correspondence Address: Baker Donelson Bearman, Caldwell & Berkowitz, PC, 555 Eleventh Street, NW, Sixth Floor, Washington, DC, 20004, US

(73) Assignee: BAYER CROPSCIENCE AG, 40789 Monheim (DE)

(21) Appl. No.: 11/917,897

(22) PCT Filed: Jun. 13, 2006
(86) PCT No.: PCT/EP2006/005650

§ 371 Date: Jun. 6, 2008

(30) Foreign Application Priority Data
Jun. 23, 2005 (DE) 102005029126.0

(51) Int. Cl. A61K 31/55; C07D 223/14; A61P 35/00

(52) U.S. Cl. 514/214.03; 540/586

(57) ABSTRACT

The present invention relates to cripowellins and synthetic derivatives thereof for treating diseases of man and also, in particular, to their use for preparing a medicament for treating cancer or other proliferative disorders in man and animal. Furthermore, the present invention relates to novel cripowellin derivatives and processes for their preparation.

The present invention relates to cripowellins and synthetic derivatives thereof for treating diseases of man and also, in particular, to their use for preparing a medicament for treating cancer or other proliferative disorders in man and animal. Furthermore, the present invention relates to novel cripowellin derivatives and processes for their preparation.

Cripowellins and semisynthetic derivatives thereof and their use for controlling animal pests are described in WO 97/34910. Furthermore, total syntheses have recently been described which affords the cripowellin skeleton (aglycon) (Moon, B. et al., Org. Lett. 7, 1031-1034, 2005; Enders, D. et al., Angew. Chem. 7, 1031-1034, 2005; Enders, D. et al., J. Org. Chem. 70, 10538-10551, 2005).

It has now been found that these compounds interact with tubulin and stabilize microtubuli.

Microtubuli play a key role in the regulation of the structure, the metabolism and the division of cells. Within the cells, tubulin is polymerized in microtubuli which form the mitotic spindle. The microtubuli are depolymerized once the mitotic spindles have served their purpose. Active compounds which interrupt polymerization or depolymerization of the microtubuli in neoplastic cells, thus inhibiting the proliferation of these cells, are among the most effective chemotherapeutic anticancer agents currently available (Jordan, M. A. und Wilson, L., Nature Rev. 4, 253-265, 2004). The best-known examples of these are discodermolides and epothilones (Nicolaou et al., Angew. Chem. 110, 2120-2153, 1998) and also paclitaxel (Taxol).

The present invention relates firstly to the use of compounds of the general formula (I)

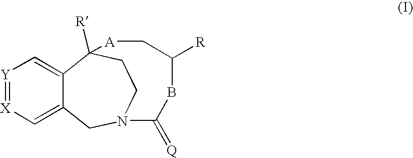

in which
R represents hydrogen or the radical —OR¹,
R′ represents hydrogen or hydroxyl,
A represents methylene, carbonyl, thiocarbonyl or the group —CH(OR²),
B represents methylene, carbonyl, thiocarbonyl or the group —CH(OR³),
Q represents oxygen or sulphur,
R¹ represents hydrogen, 2-tetrahydropyranyl, an optionally substituted glycosyl radical or one of the radicals —SO₂R⁴⁻¹, —COR⁴⁻¹, —CO₂R⁴⁻¹, —CONHR⁴⁻¹ or —CONR⁴⁻¹R⁵⁻¹,
R² represents hydrogen, 2-tetrahydropyranyl, an optionally substituted glycosyl radical or one of the radicals —SO₂R⁴⁻², —COR⁴⁻², —CO₂R⁴⁻², —CONHR⁴⁻² or —CONR⁴⁻²R⁵⁻² and
R³ represents hydrogen, 2-tetrahydropyranyl, an optionally substituted glycosyl radical or one of the radicals —SO₂R⁴⁻³, —COR⁴⁻³, —CO₂R⁴⁻³, —CONHR⁴⁻³ or —CONR⁴⁻³R⁵⁻³
   in which
   R⁴⁻¹, R⁴⁻², R⁴⁻³, R⁵⁻¹, R⁵⁻² and R⁵⁻³ independently of one another represent optionally halogen-substituted alkyl or optionally substituted aryl
      or
R¹ and R³ together represent carbonyl, thiocarbonyl or optionally methyl-substituted alkylene,
Y═X represents a group

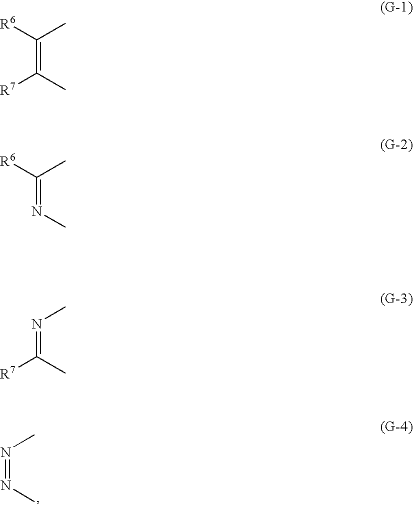

in which
R⁶ and R⁷ independently of one another represent hydrogen, halogen, hydroxyl, nitro, cyano, NR⁸R⁹, SO₂OH, SO₂NR⁸R⁹, formyl, COOH, CONR⁸R⁹, C₁₋₄-alkyl, halo-C₁₋₄-alkyl, C₁₋₄-alkylcarbonyl, halo-C₁₋₄-alkylcarbonyl, C₁₋₄-alkoxy, halo-C₁₋₄-alkoxy, C₁₋₄-alkoxycarbonyl, C₁₋₄-alkylthio, halo-C₁₋₄-alkylthio, C₁₋₄-alkylsulphinyl, halo-C₁₋₄-alkylsulphinyl, C₁₋₄-alkylsulphonyl, halo-C₁₋₄-alkylsulphonyl, C₁₋₄-alkyloxysulphonyl, C₃₋₇-cycloalkyl, optionally substituted aryl, optionally substituted aryl-C₁₋₄-alkyl, optionally substituted aryloxy, optionally substituted aryl-C₁₋₄-alkyloxy, optionally substituted hetaryl, optionally substituted hetaryloxy, optionally substituted hetaryl-C₁₋₄-alkyl or optionally substituted hetaryl-C₁₋₄-alkyloxy,
   or
R⁶ and R⁷ represent a group —O—CH₂—O—, —O—CHF—O—, —O—CF₂—O—, —O—CH₂—CH₂—O—, —O—CF₂—CF₂—O—,
R⁸ and R⁹ independently of one another represent hydrogen, C₁₋₄-alkyl, halo-C₁₋₄-alkyl, C₁₋₄-alkylcarbonyl, halo-C₁₋₄-alkylcarbonyl, C₁₋₄-alkoxy, C₁₋₄-alkoxycarbonyl, C₁₋₄-alkylsulphinyl, C₁₋₄-alkylsulphonyl, C₃₋₇-cycloalkyl, C₃₋₇-cycloalkylcarbonyl, C₃₋₇-cycloalkyl-C₁₋₂-alkyl or phenyl-C₁₋₂-alkyl,
   or
R⁸ and R⁹ as a group together with the linking nitrogen atoms form an optionally substituted five-, six- or seven-membered ring which is optionally interrupted by one or more heteroatoms for example from the group consisting of oxygen, sulphur and nitrogen, and

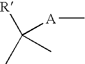

may also represent the group

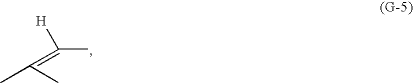

for treating diseases of man.

The glycosyl radicals in the compounds according to the invention are mono- or disaccharide radicals, in particular monosaccharides, in which optionally one or more hydroxyl groups may be substituted by acyl, alkyl or aralkyl groups. The monosaccharides may also be amino sugars in which the amino group may optionally be substituted by an acyl radical.

If appropriate, the compounds of the formula (I) may be present in different stereoisomeric forms, for example as stereoisomers of the formulae (I-A), (I-B), (I-C) and (I-D).

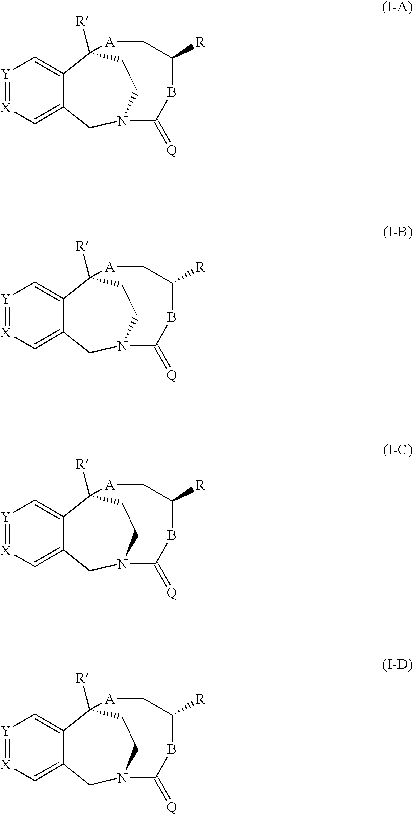

The invention also relates to the compounds of the formulae (Ib) and (Ic)—cripowellin I (also known as cripowellin A in the literature: cf., for example, Velten, R. et al. Tetrahedron Lett. 39, 1737-1740, 1998) and cripowellin II (also known as cripowellin B in the literature)-

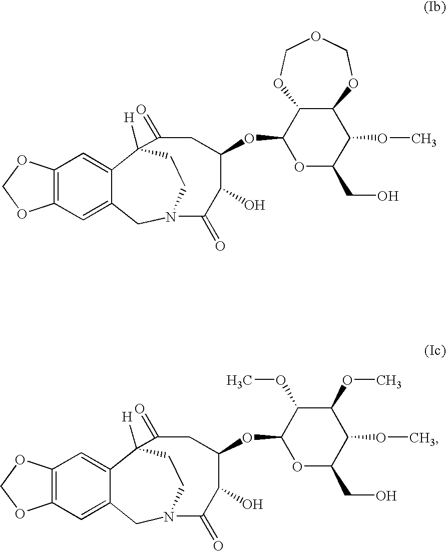

for treating diseases of man.

Preferred, particularly preferred and very particularly preferred meanings for certain radicals are indicated below.
R preferably represents the radical —OR¹.
R′ preferably represents hydrogen.
A preferably represents carbonyl.
B preferably represents the group —CH(OR³)—.
Q preferably represents oxygen.
R¹ preferably represents hydrogen, 2-tetrahydropyranyl, an optionally substituted monosaccharide radical or one of the radicals —SO₂R⁴⁻¹, —COR⁴⁻¹ or —CONHR⁴⁻¹.
R² preferably represents hydrogen, 2-tetrahydropyranyl, an optionally substituted monosaccharide radical or one of the radicals —COR⁴⁻² or —CONHR⁴⁻².
R³ preferably represents hydrogen, 2-tetrahydropyranyl, an optionally substituted monosaccharide radical or one of the radicals —COR⁴⁻³ or —CONHR⁴⁻³.
R⁴⁻¹, R⁴⁻², R⁴⁻³, R⁵⁻¹, R⁵⁻² and R⁵⁻³ independently of one another preferably represent optionally fluorine- or chlorine-substituted C₁₋₈-alkyl or optionally fluorine-, chlorine-, nitro-, C₁₋₄-alkyl- or C₁₋₄-alkoxy-substituted phenyl.
R¹ and R³ together can preferably represent carbonyl, thiocarbonyl or optionally methyl-substituted C₁₋₃-alkylene.
Y═X preferably represents the group

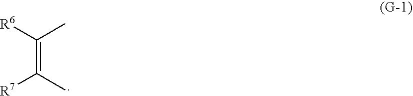

R⁶ and R⁷ independently of one another preferably represent hydrogen, fluorine, chlorine, bromine, iodine, hydroxyl, nitro, cyano, amino, N-methylamino, N,N-dimethylamino, methyl, ethyl, n-propyl, isopropyl, n-butyl, sec-butyl, isobutyl, tert-butyl, fluoromethyl, difluoromethyl, trifluoromethyl, chlorodifluoromethyl, pentafluoroethyl, n-heptafluoropropyl, isoheptafluoropropyl, isohexafluoropropyl, methylcarbonyl, ethylcarbonyl, n-propylcarbonyl, isopropylcarbonyl, n-butylcarbonyl, sec-butylcarbonyl, isobutylcarbonyl, tert-butylcarbonyl, trifluoromethylcarbonyl, pentafluoroethylcarbonyl, methoxy, ethoxy, n-propoxy, isopropoxy, n-butoxy, sec-butoxy, isobutoxy, tert-butoxy, trifluoromethoxy, chlorodifluoromethoxy, pentafluoroethoxy, methoxycarbonyl, ethoxycarbonyl, n-propoxycarbonyl, isopropoxycarbonyl, n-butoxycarbonyl, sec-butoxycarbonyl, isobutoxycarbonyl, tert-butoxycarbonyl, methylthio, ethylthio, n-propylthio, isopropylthio, n-butylthio, sec-butylthio, isobutylthio, tert-butylthio, trifluoromethylthio, trifluoromethylsulphinyl, trifluoromethylsulphonyl, cyclopropyl, cyclobutyl, cyclopentyl, cyclohexyl, optionally substituted phenyl, optionally substituted phenyl-C₁₋₄-alkyl, in particular benzyl or phenethyl, optionally substituted phenoxy, optionally substituted phenyl-C₁₋₄-alkyloxy, in particular phenylmethoxy or phenylethoxy, optionally substituted pyridyl, pyrimidyl, pyrazinyl, pyrazolyl, thiazolyl, thienyl or furyl, optionally substituted pyridyloxy, pyrimidyloxy or pyrazinyloxy, optionally substituted pyridylmethyl, pyridylethyl, pyrimidylmethyl, pyrazinylmethyl or thiazolylmethyl, optionally substituted pyridylmethoxy, pyrimidylmethoxy or pyrazinylmethoxy, which may optionally be substituted by radicals from the group consisting of fluorine, chlorine, bromine and iodine, C₁₋₄-alkyl, in particular methyl, ethyl or isopropyl, C₃₋₆-cycloalkyl, in particular cyclopropyl, cyclobutyl, cyclopentyl or cyclohexyl, C₃₋₆-cycloalkoxy, in particular cyclopropoxy, cyclobutoxy, cyclopentoxy or cyclohexoxy, C₃₋₆-cycloalkyl-C₁₋₂-alkoxy, in particular cyclopropylmethoxy or cyclopropylethoxy, C₁₋₄-haloalkyl, in particular trifluoromethyl, amino, hydroxyl, nitro, cyano, SO₂OH, COOH, formyl, C₁₋₄-alkoxy, in particular methoxy, ethoxy or isopropoxy, C₁₋₂-alkylenedioxy, in particular methylenedioxy or ethylenedioxy, C₁₋₄-haloalkoxy, in particular trifluoromethoxy or difluoromethoxy, C₁₋₄-alkylthio, in particular methylthio, C₁₋₄-alkylsulphinyl, in particular methylsulphinyl, C₁₋₄-alkylsulphonyl, in particular methylsulphonyl, C₁₋₄-haloalkylthio, in particular trifluoromethylthio, C₁₋₄-haloalkylsulphoxyl, in particular trifluoromethylsulphoxyl, C₁₋₄-haloalkylsulphonyl, in particular trifluoromethylsulphonyl, C₁₋₄-alkylamino, in particular methylamino, di-(C₁₋₄-alkyl)amino, in particular N,N-dimethylamino, N,N-diethylamino, C₁₋₄-alkylcarbonyl, in particular methylcarbonyl or ethylcarbonyl, C₃₋₆-cycloalkylcarbonyl, in particular cyclopropylcarbonyl, phenylcarbonyl, C₁₋₄-alkoxycarbonyl, in particular methoxycarbonyl or ethoxycarbonyl,
   or
   represent a group —O—CH₂—O—, —O—CHF—O—, —O—CF₂—O—, —O—CH₂—CH₂—O—, —O—CF₂—CF₂—O—.
R⁸ and R⁹ independently of one another preferably represent hydrogen, methyl, ethyl, n-propyl, isopropyl, n-butyl, isobutyl, sec-butyl or tert-butyl, trifluoromethyl, difluoromethyl, monofluoropropyl, methylcarbonyl, ethylcarbonyl, trifluoromethylcarbonyl, methoxycarbonyl, ethoxycarbonyl, n-propoxycarbonyl, methylsulphinyl, ethylsulphinyl, methylsulphonyl, ethylsulphonyl, cyclopropyl, cyclopropylmethyl, cyclopropylcarbonyl, benzyl or phenethyl.
R⁸ and R⁹ as a group together with the linking nitrogen atoms preferably represent N-pyrrolidino, N-morpholino, N-thiomorpholino, N—(N′-methyl)piperidino or a radical from the group consisting of (G-6) to (G-13)

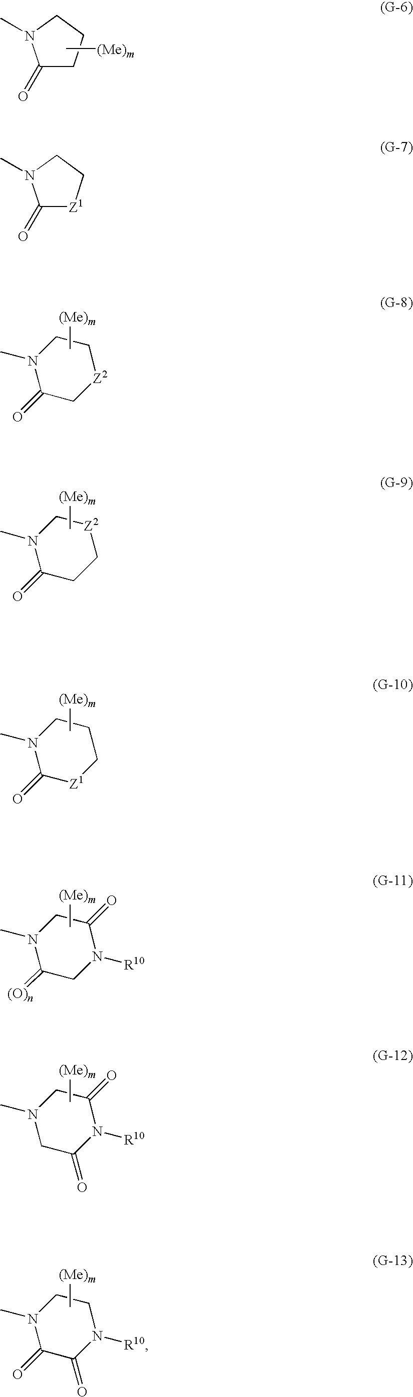

in which
Z¹ represents oxygen, methylene or N—R¹⁰,
Z² represents oxygen, sulphur, sulphinyl, sulphonyl, methylene or N—R¹⁰,
R¹⁰ represents hydrogen or C₁₋₄-alkyl, in particular methyl,
n and m independently of one another represent 0 or 1.
   R¹ particularly preferably represents an optionally substituted monosaccharide radical.
Preferred monosaccharide radicals are pyranosyl radicals, such as, for example, glucopyranosyl, galactopyranosyl or mannopyranosyl, furanosyl radicals, such as glucofuranosyl, ribofuranosyl or arabinofuranosyl, or amino sugar radicals, such as, for example, 2-amino-2-deoxy-β-D-glucopyranosyl or 2-acetylamino-2-deoxy-β-D-glucopyranosyl radicals, which are attached to the aglycon, either in α-glucosidic or β-glucosidic manner. In these radicals, one or more hydroxyl groups may optionally be substituted by acyl, alkyl or aralkyl groups. Acyl substituents which are preferred according to the invention for the monosaccharide radicals are, for example, acetyl, trichloroacetyl, benzoyl, p-nitrobenzoyl or p-methoxybenzoyl. Preferred alkyl substituents of the monosaccharide radicals are those having a low number of carbon atoms, such as methyl, ethyl, propyl or butyl. Preferred aralkyl substituents of the monosaccharide radicals are benzyl or p-methoxybenzyl groups.
Particularly preferred monosaccharide radicals are hexopyranosyl radicals, such as, for example, glucopyranosyl, galactopyranosyl or mannopyranosyl, or amino sugar radicals, such as, for example, 2-amino-2-deoxy-β-D-glucopyranosyl or 2-acetylamino-2-deoxy-β-D-glucopyranosyl radicals, which are attached to the aglycon either in α-glycosidic or β-glycosidic manner.
Very particularly preferred monosaccharide radicals are glucopyranosyl, galactopyranosyl, mannopyranosyl or 2-amino-2-deoxy-β-D-glucopyranosyl or 2-acetylamino-2-deoxy-β-D-glucopyranosyl, which are attached to the aglycon either in α-glycosidic or β-glycosidic manner. In these radicals, one or more hydroxyl groups may optionally be substituted by acetyl, trichloroacetyl, benzoyl, p-nitrobenzoyl, p-methoxybenzoyl, methyl, ethyl, propyl, butyl, benzyl or p-methoxybenzyl.
R³ particularly preferably represents hydrogen.
R⁴⁻¹, R⁴⁻² and R⁴⁻³ independently of one another particularly preferably represent methyl, trifluoromethyl, ethyl or optionally fluorine-, chlorine-, nitro-, methyl-, ethyl-, methoxy-substituted phenyl.
R¹ and R³ together may also particularly preferably represent carbonyl, thiocarbonyl or one of the groups —CH₂—, —CH(CH₃)—, —C(CH₃)₂—, —(CH₂)₂—, —C H(CH₃)CH₂— or —CH(CH₃)CH(CH₃)—
R⁶ and R⁷ independently of one another particularly preferably represent hydrogen, fluorine, chlorine, nitro, cyano, N,N-dimethylamino, methyl, ethyl, trifluoromethyl, methoxy, ethoxy, trifluoromethoxy, methoxycarbonyl, ethoxycarbonyl, tert-butoxycarbonyl, methylthio, trifluoromethylthio, trifluoromethylsulphinyl, trifluoromethylsulphonyl, cyclopropyl or represent the group —O—CH₂—O—.
R⁸ and R⁹ independently of one another particularly preferably represent hydrogen, methyl, ethyl, trifluoromethyl, difluoromethyl, methylcarbonyl, trifluoromethylcarbonyl, methoxycarbonyl, ethoxycarbonyl, methylsulphinyl, methylsulphonyl, cyclopropyl, cyclopropylmethyl, cyclopropylcarbonyl or benzyl.
R⁸ and R⁹ as grouped together with the linking nitrogen atoms particularly preferably represent N-pyrrolidino, N-morpholino, N-thiomorpholino or N—(N′-methyl)piperidino.
R⁶ and R⁷ independently of one another very particularly preferably represent hydrogen, fluorine, chlorine, nitro, N,N-dimethylamino, methyl, trifluoromethyl, methoxy, trifluoromethoxy, methoxycarbonyl, ethoxycarbonyl, tert-butoxycarbonyl, methylthio, trifluoromethylthio, trifluoromethylsulphinyl, trifluoromethylsulphonyl or represent the group —O—CH₂—O—.
R⁸ and R⁹ independently of one another very particularly preferably represent methyl, methoxycarbonyl or cyclopropylcarbonyl.

Particularly preferred compounds of the formula (I) are the stereoisomers of the formulae (I-A-1) and (I-B-1).

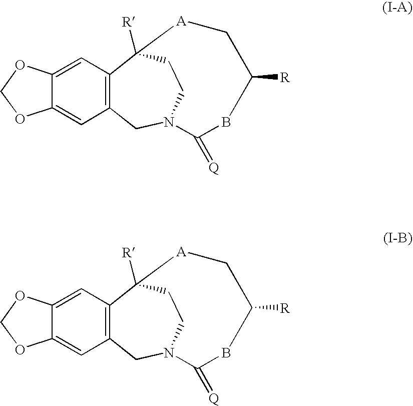

Very particularly preferred compounds of the formula (I) are stereoisomers of the formula (I-A-1)

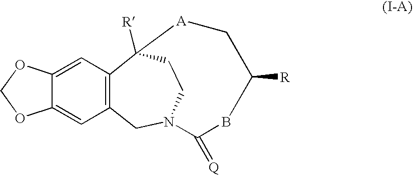

The general or preferred radical definitions or illustrations given above apply both to the end products and, correspondingly, to starting materials and intermediates. These radical definitions may also be combined with one another as desired, i.e. including combinations between the respective preferred ranges.

Preference according to the invention is given to compounds of the formula (I) which contain a combination of the meanings given above as being preferred.

Particular preference according to the invention is given to compounds of the formula (I) which contain a combination of the meanings given above as being particularly preferred.

Very particular preference according to the invention is given to compounds of the formula (I) which contain a combination of the meanings given above as being very particularly preferred.

The present invention also relates to novel compounds of the general formula (I)

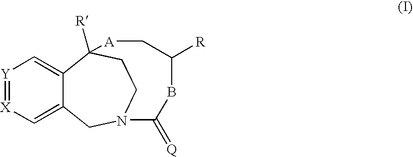

in which
A, B, R, R′, Q, X and Y are as defined above, with the exception that R⁶ and R⁷ do not represent the group —O—CH₂—O—.

The compounds of the general formula (I) according to the invention can also be present in the form of an acid addition salt. Acids which can be used for salt formation are inorganic acids, such as hydrochloric acid, hydrobromic acid, nitric acid, sulphuric acid, phosphoric acid, or organic acids, such as formic acid, acetic acid, propionic acid, malonic acid, oxalic acid, fumaric acid, adipic acid, stearic acid, tartaric acid, oleic acid, methanesulphonic acid, benzenesulphonic acid or toluenesulphonic acid.

Suitable salts of the compounds of the general formula (I) which may be mentioned are customary nontoxic salts, i.e. salts with various bases and salts with added acids. Preference is given to salts with inorganic bases, such as alkali metal salts, for example sodium, potassium or caesium salts, alkaline earth metal salts, for example calcium or magnesium salts, ammonium salts, salts with organic bases and also with organic amines, for example triethylammonium, pyridinium, picolinium, ethanolammonium, triethanolammonium, dicyclohexylammonium or N,N′-dibenzylethylenediammonium salts, salts with inorganic acids, for example hydrochlorides, hydrobromides, dihydrosulphates or trihydrophosphates, salts with organic carboxylic acids or organic sulphonic acids, for example formates, acetates, trifluoroacetates, maleates, tartrates, methanesulphonates, benzenesulphonates or para-toluenesulphonates, salts with basic amino acids or acidic amino acids, for example arginates, aspartates or glutamates.

The compounds of the formula (I) according to the invention may, if appropriate, exist in stereoisomeric forms which are either like image and mirror image (enantiomers) or which are not like image and mirror image (diastereomers). The present invention provides both the enanatiomers and the diastereomers, and their respective mixtures. The racemic forms, like the diastereomers, can be separated in a known manner into the stereoisomerically uniform components. If appropriate, the isomers may be converted into one another by processes known per se. The invention relates both to the pure isomers and to the isomer mixtures.

The present invention furthermore relates to processes for preparing the novel compounds of the general formula (I)

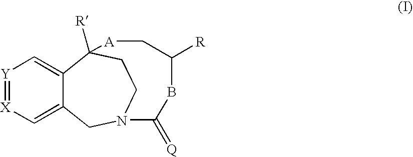

in which

A, B, R, R′, Q, X and Y are as defined above, with the exception that R⁶ and R⁷ do not represent the group —O—CH₂—O—, wherein

a) compounds of the general formula (II)

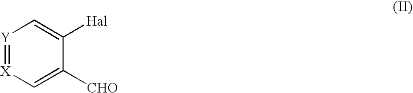

in which
Hal represents a halogen, preferably bromine, and
X and Y are as defined above
are, in a first reaction step, reacted with 3-buten-1-amine, if appropriate in the presence of a diluent and if appropriate in the presence of an acidic reaction auxiliary and if appropriate with removal of water, to give the corresponding compounds of the general formula (III)

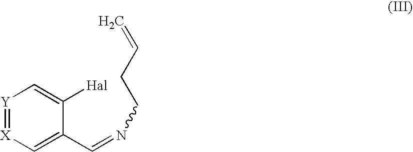

in which
Hal represents a halogen, preferably bromine, and
X and Y are as defined above,
and these compounds are then, in a second reaction step, if appropriate in situ and if appropriate in the presence of a diluent and if appropriate in the presence of a hydrogenating agent, converted into compounds of the general formula (IV)

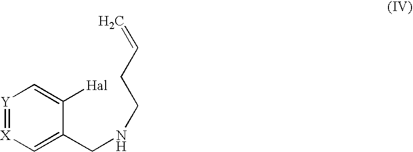

in which
Hal represents a halogen, preferably bromine, and
X and Y are as defined above,
and these compounds are then, in a third reaction step, if appropriate in the presence of a diluent and if appropriate in the presence of a basic reaction auxiliary, reacted with compounds of the general formula (V)

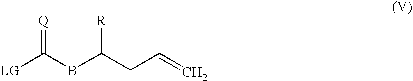

in which
LG is a nucleofugic leaving group, if appropriate generated in situ, and
B, R and Q are as defined above, where Q in the general formula (V) preferably represents oxygen,
if appropriate in the presence of a diluent and if appropriate in the presence of a basic reaction auxiliary, to give compounds of the general formula (VI),

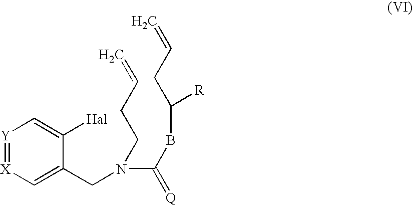

in which
Hal represents a halogen, preferably bromine, and
B, R, Q, X and Y are as defined above, where Q in the general formula (VI) preferably represents oxygen,
and these compounds are, in a fourth reaction step, under the reaction conditions of a metathesis reaction, if appropriate in the presence of a suitable catalyst and if appropriate in the presence of a diluent, converted into compounds of the general formula (VII)

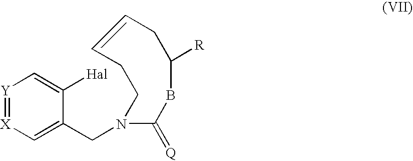

in which
Hal represents halogen, preferably bromine, and
B, R, Q, X and Y are as defined above, where Q in the general formula (VII) preferably represents oxygen,
and these compounds are then, in a fifth reaction step, under the reaction conditions of a Heck reaction, if appropriate in the presence of suitable noble metal salts and if appropriate in the presence of a suitable catalyst and if appropriate in the presence of a diluent, converted into compounds of the general formula (Ia), and/or
b) the compounds of the general formula (Ia)

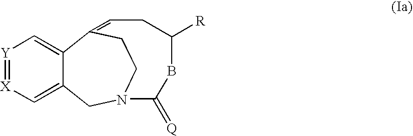

in which
B, R, Q, X and Y are as defined above
are, in a first reaction step, by oxidation of the C═C double bond, if appropriate in the presence of a diluent, converted into compounds of the general formula (I)

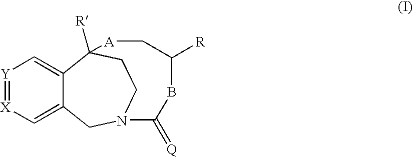

in which
R′ represents hydroxyl,
A represents CH—OH, and
B, R, Q, X and Y are as defined above,
and these compounds are then, in a second reaction step, by oxidation of the CH—OH group A in the presence of a diluent, converted into compounds of the general formula (I)
in which
R′ represents hydroxyl, and
A, B, R, Q, X and Y are as defined above,
and these compounds are then, in a third reaction step, in the presence of a suitable salt of a lanthanoid metal, in particular samarium(II) iodide, if appropriate in the presence of a diluent, converted into compounds of the general formula (I)
in which
R′ represents hydrogen, and
A, B, R, Q, X and Y are as defined above.

If, to prepare the novel compounds of the general formula (Ia), use is made, for example, of 2-bromo-5-fluorobenzaldehyde as compound of the formula (II), of 3-buten-1-amine and, for example, of (4S,5R)-5-allyl-2,2-dimethyl-1,3-dioxolane-4-carboxylic acid as compound of the general formula (V) in the presence of 1-ethyl-2-fluoropyridinium tetrafluoroborate (FEP), the preparation process a), which entails five reaction steps, can be represented by reaction scheme I below:

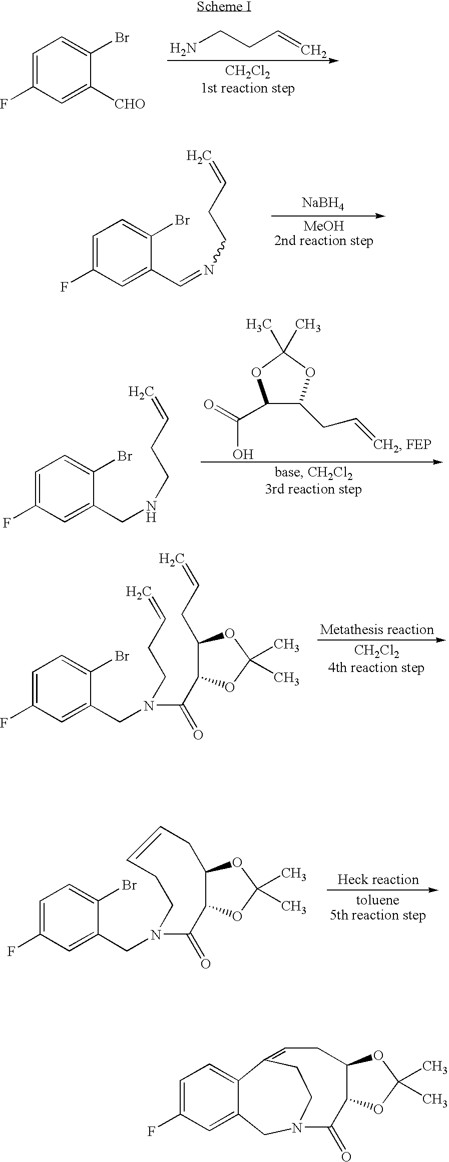

The preparation process a) is based on total synthesis of the cripowellin skeleton recently published by Enders et al. (J. Org. Chem. 70, 10538-10551, 2005).

Surprisingly and according to the invention, it has been found that the preparation of the cripowellin skeleton by the preparation process described above provides access to novel, hitherto unknown compounds of the general formula (I) and is thus not limited to the known substituent pattern (X—Y≡O—CH₂—O—) of naturally occurring cripowellins A and B.

The formula (II) provides a general definition of the compounds required as starting materials for carrying out the process a) according to the invention.

In this formula (II), X and Y preferably represent those radicals which have already been mentioned in connection with the description of the novel compounds according to the invention of the general formula (I) as preferred substituents.

Some of the compounds of the general formula (II) can be obtained from commercially available compounds, for example optionally 4-substituted, 5-substituted or 4,5-disubstituted 2-bromobenzaldehydes and nitrogenous aldehydes (for example 3-bromo-4-pyridinaldehyde (X═CH, Y═N): Corey, E. J. et al., Tetrahedron Letters 24, 3291-3294, 1983; Mandal, A. B. et al., Tetrahedron Letters 46, 6033-6036, 2005; 4-bromo-3-pyridinaldehyde (X═N, Y═CH): Phuan, P.-W.; Kozlowski, M. C., Science of Synthesis 15, 947-985, 2005; Numata, A. et al., Synthesis 2, 306-311, 1999, which may optionally be substituted, using methods known from the literature (cf., for example, aromatic aldehydes from methylarenes: Houben-Weyl, Methoden der Organischen Chemie [Methods of organic chemistry], volume VII/1, 211; by the Gattermann-Koch synthesis: Houben-Weyl, Methoden der Organischen Chemie, volume VII/1, 16; by the Sommelet reaction: Houben-Weyl, Methoden der Organischen Chemie, volume VII/1, 194; cf. also C. Ferri “Reaktionen der Organischen Synthese” [Reactions of organic synthesis]; Georg Thieme Verlag Stuttgart, 1978, p. 415 and the literature cited therein).

Another compound required as starting material for the process a) according to the invention is the commercially available 3-buten-1-amine.

Further compounds (V) required as starting materials for the process a) according to the invention are unsaturated carboxylic acid derivatives. Some of these are commercially available, such as, for example, 5-hexenoic acid, or they can be obtained by methods known from the literature, such as, for example, (4S,5R)-5-allyl-2,2-dimethyl-1,3-dioxolane-4-carboxylic acid (Enders, D. et al., J. Org. Chem. 70, 10538-10551, 2005; Enders, D. et al., Angew. Chem., Intern. Ed. 44, 3766-3769, 2005) or 3S-hydroxy-5-hexenoic acid (Maddrell, S., Tetrahedron Letters 37, 6001-6004, 1996).

When preparing further derivatives from the compounds according to the invention of the general formula (V) in which R represents, for example, hydroxyl and B represents a group CHOH, it is advantageous to use suitable protective groups (SG) (scheme II). Substituted methyl ethers and ethers, substituted ethyl ethers, substituted benzyl ethers, silyl ethers, esters, carbonates or sulphonates, for example, are known as protective groups for hydroxyl groups (cf. Greene T. W., Wuts P. G. W. in Protective Groups in Organic Synthesis; John Wiley & Sons, Inc. 1999, “Protection for the hydroxyl group including 1,2- and 1,3-diols”).

Protective groups of the substituted methyl ether type which may be mentioned are, for example: methoxymethyl ether (MOM), methylthiomethyl ether (MTM), (phenyldimethylsilyl)methoxy-methyl ether (SNOM-OR), benzyloxymethyl ether (BOM-OR), para-methoxybenzyloxymethyl ether (PMBM-OR), para-nitrobenzyloxymethyl ether, ortho-nitrobenzyloxymethyl ether (NBOM-OR), (4-methoxyphenoxy)methyl ether (p-AOM-OR), guaiacolmethyl ether (GUM-OR), tert-butoxymethyl ether, 4-pentyloxymethyl ether (POM-OR), silyloxymethyl ether, 2-methoxyethoxymethyl ether (MEM-OR), 2,2,2-trichloroethoxymethyl ether, bis(2-chloroethoxy)methyl ether, 2-(trimethylsilyl)ethoxymethyl ether (SEM-OR), methoxymethyl ether (MM-OR).

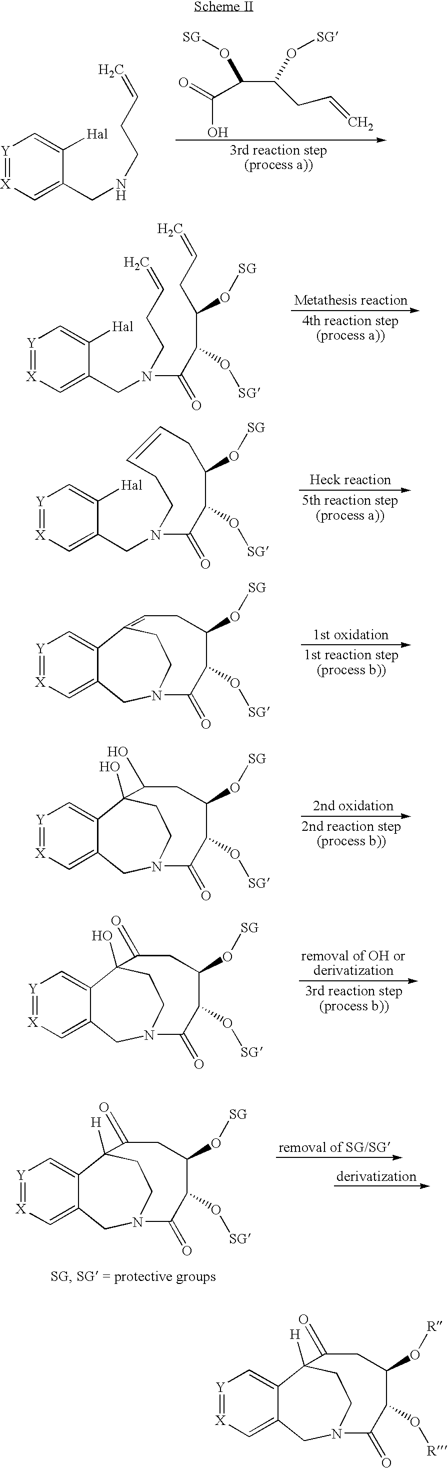

Protective groups (SG) of the substituted ethyl ether type which may be mentioned are, for example: 1-ethoxyethyl ether (EE-OR), 1-(2-chloroethoxy)ethyl ether (CEE-OR), 1-[2-(trimethyl-silyl)ethoxy]ethyl ether (SEE-OR), 1-methyl-1-methoxyethyl ether (MIP-OR), 1-methyl-1-benzyloxyethyl ether (MBE-OR), 1-methyl-1-benzyloxy-2-fluoroethyl ether (MIP-OR), 1-methyl-1-phenoxyethyl ether, 2,2,2-trichloroethyl ether, 1,1-dianisyl-2,2,2-trichloroethyl ether (DATE-OR), 1,1,1,3,3,3-hexafluoro-2-phenylisopropyl ether (HIP-OR), 2-trimethylsilylethyl ether, 2-(benzylthio)ethyl ether, 2-(phenylselenyl)ethyl ether. Further protective groups (SG) of the ether type which may be mentioned are, for example: tetrahydropyranyl ether (THP-OR), 3-bromotetrahydropyranyl ether (3-BrTHP-OR), tetrahydrothiopyranyl ether, 1-methoxycyclo-hexyl ether, 2- and 4-picolyl ether, 3-methyl-2-picolyl-N-oxido ether, 2-quinolinylmethyl ether (Qm-OR), 1-pyrenylmethyl ether, dipenylmethyl ether (DPM-OR), para, para′-dinitrobenzhydryl ether (DNB-OR), 5-dibenzosuberyl ether, triphenylmethyl ether (Tr-OR), alpha-naphthyldiphenylmethyl ether, para-methoxyphenyldiphenylmethyl ether (MMTrOR), di(para-methoxyphenyl)phenylmethyl ether (DMTr-OR), tri(para-methoxyphenyl)phenylmethyl ether (TMTr-OR), 4-(4′-bromophenacyloxy)phenyldiphenylmethyl ether, 4,4′,4′″-tris(4,5-dichloro-phthalimidophenyl)methyl ether (CPTr-OR), 4,4′,4″-tris(benzoyloxyphenyl)methyl ether (TBTr-OR), 4,4′-dimethoxy-3″-[N-(imidazolylmethyl)]trityl ether (IDTr-OR), 4,4′-dimethoxy-3″-[N-(imidazolylethyl)carbamoyl]trityl ether (IETr-OR), 1,1-bis(4-methoxyphenyl)-1′-pyrenylmethyl ether (Bmpm-OR), 9-anthryl ether, 9-(9-phenyl)xanthenyl ether (Pixyl-OR), 9-(9-phenyl-10-oxo)anthryl ether (tritylon ether), 4-methoxytetrahydropyranyl ether (MTHP-OR), 4-methoxytetrahydrothiopyranyl ether, 4-methoxytetrahydrothiopyranyl S,S-dioxide, 1-[(2-chloro-4-methyl)phenyl]-4-methoxypiperidin-4-yl ether (CTMP-OR), 1-(2-fluorophenyl)-4-methoxy-piperidin-4-yl ether (Fpmp-OR), 1,4-dioxan-2-yl ether, tetrahydrofuranyl ether, tetrahydro-thiofuranyl ether, 2,3,3a,4,5,6,7,7a-octahydro-7,8,8-trimethyl-4,7-methanebenzofuran-2-yl ether (MBF-OR), tert-butyl ether, allyl ether, propargyl ether, para-chlorophenyl ether, para-methoxyphenyl ether, para-nitrophenyl ether, para-2,4-dinitrophenyl ether (DNP-OR), 2,3,5,6-tetrafluoro-4-(trifluoromethyl)phenyl ether, benzyl ether (Bn-OR). Protective groups (SG) of the substituted benzyl ether type which may be mentioned are, for example: para-methoxybenzyl ether (MPM-OR), 3,4-dimethoxybenzyl ether (DMPM-OR), ortho-nitrobenzyl ether, para-nitrobenzyl ether, para-halobenzyl ether, 2,6-dichlorobenzyl ether, para-aminoacylbenzyl ether (PAB-OR), para-azidobenzyl ether (Azb-OR), 4-azido-3-chlorobenzyl ether, 2-trifluoromethylbenzyl ether, para-(methylsulphinyl)benzyl ether (Msib-OR). Protective groups (SG) of the silyl ether type which may be mentioned are, for example: trimethylsilyl ether (TMS-OR), triethylsilyl ether (TES-OR), triisopropylsilyl ether (TIPS-OR), dimethylisopropylsilyl ether (IPDMS-OR), diethylisopropylsilyl ether (DEIPS-OR), dimethylhexylsilyl ether (TDS-OR), tert-butyldimethylsilyl ether (TBDMS-OR), tert-butyldiphenylsilyl ether (TBDPS-OR), tribenzylsilyl ether, tri-para-xylylsilyl ether, triphenylsilyl ether (TPS-OR), diphenylmethylsilyl ether (DPMS-OR), di-tert-butylmethylsilyl ether (DTBMS-OR), tris(trimethylsilyl)silyl ether (sisyl ether), (2-hydroxystyryl)dimethylsilyl ether (HSDMS-OR), (2-hydroxystyryl)diisopropylsilyl ether (HSDIS-OR), tert-butylmethoxyphenylsilyl ether (TBMPS-OR), tert-butoxydiphenylsilyl ether (DPTBOS-OR). Protective groups (SG) of the ester type which may be mentioned are, for example: formate ester, benzoylformate ester, acetate ester (Ac-OR), chloroacetate ester, dichloroacetate ester, trichloroacetate ester, trifluoroacetate ester (TFA-OR), methoxyacetate ester, triphenylmethoxyacetate ester, phenoxyacetate ester, para-chlorophenoxyacetate ester, phenylacetate ester, diphenylacetate ester (DPA-OR), nicotinate ester, 3-phenylpropionate ester, 4-pentoate ester, 4-oxopentoate ester (levulinate) (Lev-OR), 4,4-(ethylenedithio)pentanoate ester (LevS-OR), 5-[3-bis(4-methoxyphenyl)hydroxymethoxyphenoxy]levulinate ester, pivaloate ester (Pv-OR), 1-adamantanoate ester, crotonate ester, 4-methoxycrotonate ester, benzoate ester (Bz-OR), para-phenylbenzoate ester, 2,4,6-trimethylbenzoate ester (mesitoate), 4-(methylthiomethoxy)butyrate ester (MTMB-OR), 2-(methylthiomethoxymethyl)benzoate ester (MTMT-OR). Protective groups (SG) of the ester type which may be mentioned are, for example: methyl carbonate, methoxymethyl carbonate, 9-fluorenylmethyl carbonate (Fmoc-OR), ethyl carbonate, 2,2,2-trichloroethyl carbonate (Troc-OR), 1,1-dimethyl-2,2,2-trichloroethyl carbonate (TCBOC-OR), 2-(trimethylsilyl)ethyl carbonate (TMSEC-OR), 2-(phenylsulphonyl)ethyl carbonate (Psec-OR), 2-(triphenylphosphonio)ethyl carbonate (Peoc-OR), tert-butyl carbonate (Boc-OR), isobutyl carbonate, vinyl carbonate, allyl carbonate (Alloc-OR), para-nitrophenyl carbonate, benzyl carbonate (Z-OR), para-methoxybenzyl carbonate, 3,4-dimethoxybenzyl carbonate, ortho-nitrobenzyl carbonate, para-nitrobenzyl carbonate, 2-dansylethyl carbonate (Dnseoc-OR), 2-(4-nitrophenyl)ethyl carbonate (Npeoc-OR), 2-(2,4-dinitrophenyl)ethyl carbonate (Dnpeoc). Protective groups (SG) of the sulphate type which may be mentioned are, for example: allylsulphonate (Als-OR), methanesulphonate (Ms-OR), benzylsulphonate, tosylate (Ts-OR), 2-[(4-nitrophenyl)ethyl]sulphonate (Npes-OR).

When preparing further derivatives from the abovementioned compounds of the general formula (I) it may well be advantageous to use, initially, compounds of the general formula (V) in which R represents, for example, hydroxyl and B represents a group CHOH, a suitable protective group, for example one of the protective groups mentioned above. Here, it may also be expedient to use two different protective groups SG and SG′, which should correspondingly be compatible with one another, i.e. they should be removable selectively and independently of one another. It is then possible to carry out the derivatization, for example a glycosidation by chemical synthesis or by microbial bioconversion (cf. also the introduction of substituents into spinosyn derivatives; for example WO 03/010155 A1).

In general, it is advantageous to carry out the preparation process a) according to the invention in the presence of diluents. Diluents are advantageously employed in such an amount that the reaction mixture remains readily stirrable during the entire process. Suitable diluents for carrying out the process a) according to the invention are all inert organic solvents.

Examples which may be mentioned are: halogenated hydrocarbons, in particular chlorinated hydrocarbons, such as tetraethylene, tetrachloroethane, dichloropropane, methylene chloride, dichlorobutane, chloroform, carbon tetrachloride, trichloroethane, trichloroethylene, pentachloroethane, difluorobenzene, 1,2-dichloroethane, chlorobenzene, bromobenzene, dichlorobenzene, chlorotoluene, trichlorobenzene; alcohols, such as methanol, ethanol, isopropanol, butanol; ethers such as ethyl propyl ether, methyl tert-butyl ether, n-butyl ether, anisole, phenetol, cyclohexyl methyl ether, dimethyl ether, diethyl ether, dipropyl ether, diisopropyl ether, di-n-butyl ether, diisobutyl ether, diisoamyl ether, ethylene glycol dimethyl ether, tetrahydrofuran, dioxane, dichlorodiethyl ether and polyethers of ethylene oxide and/or propylene oxide; amines, such as trimethylamine, triethylamine, tripropylamine, tributylamine, N-methylmorpholine, pyridine and tetramethylenediamine; nitrated hydrocarbons, such as nitromethane, nitroethane, nitropropane, nitrobenzene, chloronitrobenzene, o-nitrotoluene; nitriles, such as acetonitrile, propionitrile, butyronitrile, isobutyronitrile, benzonitrile, m-chlorobenzonitrile, and also compounds such as tetrahydrothiophene dioxide and dimethyl sulphoxide, tetramethylene sulphoxide, dipropyl sulphoxide, benzyl methyl sulphoxide, diisobutyl sulphoxide, dibutyl sulphoxide, diisoamyl sulphoxide; sulphones, such as dimethyl sulphone, diethyl sulphone, dipropyl sulphone, dibutyl sulphone, diphenyl sulphone, dihexyl sulphone, methyl ethyl sulphone, ethyl propyl sulphone, ethyl isobutyl sulphone and pentamethylene sulphone; aliphatic, cycloaliphatic or aromatic hydrocarbons, such as pentane, hexane, heptane, octane, nonane and industrial hydrocarbons, for example white spirits with components having boiling points in the range of, for example, from 40° C. to 250° C., cymene, petroleum fractions having a boiling point range of from 70° C. to 190° C., cyclohexane, methylcyclohexane, petroleum ethers, ligroin, octane, benzene, toluene, chlorobenzene, bromobenzene, nitrobenzene, xylene; esters, such as methyl acetate, ethyl acetate, butyl acetate, isobutyl acetate, and also dimethyl carbonate, dibutyl carbonate, ethylene carbonate; amides, such as hexamethylene phosphoric triamide, formamide, N-methylformamide, N,N-dimethylformamide, N,N-dipropylformamide, N,N-dibutylformamide, N-methylpyrrolidine, N-methylcaprolactam, 1,3-dimethyl-3,4,5,6-tetrahydro-2(1H)-pyrimidine, octylpyrrolidone, octylcaprolactam, 1,3-dimethyl-2-imidazolinedione, N-formylpiperidine, N,N′-1,4-diformylpiperazine; ketones, such as acetone, acetophenone, methyl ethyl ketone, methyl butyl ketone.

It is, of course, also possible to use mixtures of the solvents and diluents mentioned for the process according to the invention.

However, preferred diluents for carrying out the first reaction step in the process a) according to the invention are chlorinated hydrocarbons, such as tetraethylene, tetrachloroethane, dichloropropane, methylene chloride, dichlorobutane, chloroform, carbon tetrachloride, trichloroethane, trichloroethylene, pentachloroethane, difluorobenzene, 1,2-dichloroethane, chlorobenzene, bromobenzene, dichlorobenzene, chlorotoluene, trichlorobenzene, in particular dichloromethane.

The reaction of compounds of the general formula (II) according to the first reaction step in preparation process a) is carried out by reacting the compounds of the general formula (II) in the presence of 3-buten-1-amine, if appropriate in the presence of an acidic auxiliary and in one of the diluents stated.

The reaction time is from 10 minutes to 48 hours. The reaction is carried out at temperatures between −110° C. and +200° C., preferably between +10° C. and 180° C., particularly preferably between 15° C. and 100° C. The reaction is preferably carried out under reaction conditions allowing water to be separated off or removed, using, for example, a water separator or a dehydrating agent, such as, for example, a molecular sieve. In the first reaction step in preparation process a), preference is given to using a molecular sieve 4 Å.

In principle, the reaction can be carried out under atmospheric pressure. The reaction is preferably carried out under atmospheric pressure or under pressures of up to 15 bar and, if appropriate, under an atmosphere of protective gas (nitrogen, helium or argon).

After the reaction has ended, the entire reaction mixture is concentrated. The resulting compounds of the general formula (III) can be purified in a customary manner by recrystallization, vacuum distillation or column chromatography. However, alternatively, the compounds of the general formula (III) can be used without further purification for carrying out the second reaction step (cf. Preparation Examples).

The reaction of compounds of the general formula (III) according to the second reaction step in preparation process a) is carried out by reacting the compounds of the general formula (III) in the presence of a hydrogenating agent in one of the diluents mentioned further above.

However, preferred diluents for carrying out the second reaction step in the preparation process a) according to the invention are alcohols, such as methanol, ethanol, isopropanol, butanol, in particular methanol.

Suitable for hydrogenating the compounds of the general formula (III) are various hydrogenating agents, such as, for example, alkali metal hydrides, in particular sodium borohydride (NaBH₄), lithium aluminium hydride (LiAlH₄), lithium triethylborohydride (Li[Et₃BH]), lithium tri-sec-butylborohydride (Li[sec-Bu₃BH]), sodium bis(2-methoxyethoxy)aluminium hydride, alkylaluminium hydride, in particular diisobutylaluminium hydride (DIBAL-H), or tetramethylammonium triacetoxyborohydride, inter alia (cf. H. de Koning, W. N. Speckamp, Houben Weyl, Methoden der Organischen Chemie [Methods of organic chemistry], volume E 21, p. 1953 and the literature cited therein). It is, of course, also possible to use a “borohydride resin”, for example “borohydride on Amberlite® IRA-406”, for the hydrogenation (cf. Sande, A. R. et al., Tetrahedron Lett. 25, 3501, 1984).

Preferred for carrying out the hydrogenation is the use of alkali metal hydrides, in particular sodium borohydride (NaBH₄) and lithiumaluminium hydride (LiAlH₄).

The reaction time is from 10 minutes to 48 hours. The reaction is carried out at temperatures between −10° C. and +200° C., preferably between +10° C. and 140° C., particularly preferably between 15° C. and 80° C. The reaction is preferably carried out under atmospheric pressure or under pressures of up to 15 bar and, if appropriate, under an atmosphere of protective gas (nitrogen, helium or argon).

After the reaction has ended, the entire reaction mixture is concentrated. The resulting compounds of the general formula (IV), N-(2-bromo-5-fluorobenzyl)-N-but-3-en-1-ylamine, can be purified in a customary manner by recrystallization, vacuum distillation or column chromatography. However, alternatively, the compounds of the general formula (IV) can be used without further purification for carrying out the third reaction step (cf. Preparation Examples).

The reaction of compounds of the general formula (IV) according to the third reaction step in preparation process a) is carried out by reacting the compounds of the general formula (IV) with the compounds, mentioned further above, of the general formula (V) in the presence of a coupling agent for the carboxylic acid and, if appropriate, in the presence of a basic reaction auxiliary, in one of the diluents mentioned further above.

Suitable coupling agents for carrying out the preparation process a) are all coupling agents suitable for forming an amide bond (cf., for example, Houben-Weyl, Methoden der Organischen Chemie, volume 15/2; Bodansky et al., Peptide Synthesis 2nd ed. (Wiley & Sons, New York 1976) or Gross, Meienhofer, The Peptides: Analysis, Synthesis, Biology (Academic Press, New York 1979). Preference is given to using the following methods: the activated ester method using pentachlorophenol (Pcp) or pentafluorophenol (Pfp), N-hydroxysuccinimide (HOSu), N-hydroxy-5-norbornene-2,3-dicarboxamide (HONB), 1-hydroxybenzotriazole (HOBt) or 3-hydroxy-4-oxo-3,4-dihydro-1,2,3-benzotriazine as alcohol component, the coupling with carbodiimides, such as dicyclohexylcarbodiimide (DCCI), using the DCC additive process, or using n-propanephosphonic anhydride (PPA), and the mixed-anhydride method using pivaloyl chloride, ethyl chloroformate (EEDQ) and isobutyl chloroformate (IIDQ) or the coupling with phosphonium reagents, such as benzotriazol-1-yloxytris(dimethylaminophosphonium) hexafluorophosphate (BOP), bis(2-oxo-3-oxazolidinyl)phosphinic chloride (BOP-Cl), benzotriazol-1-yltrispyrrolidinophosphonium hexafluorophosphate (PyBOP®), bromotrispyrrolidinophosphonium hexafluorophosphate (PyBroP®), or using phosphonic acid reagents, such as diethyl cyanophosphonate (DEPC) and diphenylphosphoryl azide (DPPA), uronium reagents, such as 2-(1H-benzotriazol-1-yl)-1,1,3,3-tetramethyluronium tetrafluoroborate (TNTU), 2-(2-oxo-1(2H)-pyridyl)-1,1,3,3-bispenta-methylenetetramethyluronium tetrafluoroborate (TOPPipU), O—(N-succinimidyl)-1,1,3,3-tetramethyluronium tetrafluoroborate (TSTU), or 2-(1H-benzotriazol-1-yl)-1,1,3,3-tetramethyluronium hexafluorophosphate (HBTU), or reagents of the onium type, such as, for example, 1-ethyl-2-fluoropyridinium tetrafluoroborate (FEP).

A preferred activating agent for the carboxylic acid is, for example, 1-ethyl-2-fluoropyridinium tetrafluoroborate (FEP) (cf. Li, P., Xu, J. C., J. Peptide Res. 58, 129-139, 2001).

However, preferred diluents for carrying out the third reaction step in the process a) according to the invention are chlorinated hydrocarbons, such as tetraethylene, tetrachloroethane, dichloropropane, methylene chloride, dichlorobutane, chloroform, carbon tetrachloride, trichloroethane, trichloroethylene, pentachloroethane, difluorobenzene, 1,2-dichloroethane, chlorobenzene, bromobenzene, dichlorobenzene, chlorotoluene, trichlorobenzene, in particular dichloromethane.

Suitable basic reaction auxiliaries for carrying out the process according to the invention are all suitable acid binders, such as amines, in particular tertiary amines, and also alkali metal and alkaline earth metal compounds.

Examples which may be mentioned are the hydroxides, hydrides, oxides and carbonates of lithium, sodium, potassium, magnesium, calcium and barium, furthermore other basic compounds, such as amidine bases or guanidine bases, such as 7-methyl-1,5,7-triazabicyclo[4.4.0]dec-5-ene (MTBD); diazabicyclo[4.3.0]nonene (DBN), diazabicyclo[2.2.2]octane (DABCO), 1,8-diazabicyclo[5.4.0]-undecene (DBU), cyclohexyltetrabutylguanidine (CyTBG), cyclohexyltetramethylguanidine (CyTMG), N,N,N,N-tetramethyl-1,8-naphthalenediamine, pentamethylpiperidine, tertiary amines, such as triethylamine, trimethylamine, tribenzylamine, triisopropylamine, tributylamine, tricyclohexylamine, triamylamine, trihexylamine, N,N-diisopropylethylamine, N,N-dimethyl-aniline, N,N-dimethyltoluidine, N,N-dimethyl-p-aminopyridine, N-methylpyrrolidine, N-methyl-piperidine, N-methylimidazole, N-methylpyrazole, N-methylmorpholine, N-methylhexamethylene-diamine, pyridine, 4-pyrrolidinopyridine, 4-dimethylaminopyridine, quinoline, α-picoline, β-picoline, isoquinoline, pyrimidine, acridine, N,N,N′,N′-tetramethylenediamine, N,N,N′,N′-tetraethylenediamine, quinoxaline, N-propyldiisopropylamine, N-ethyldiisopropylaamine, N,N′-dimethylcyclohexylamine, 2,6-lutidine, 2,4-lutidine or triethyldiamine.

Preference is given to using tertiary amines, such as triethylamine, trimethylamine, tribenzylamine, triisopropylamine, tributylamine, tricyclohexylamine, triamylamine, N,N-dimethyltoluidine, N,N-dimethyl-p-aminopyridine, N-methylpyrrolidine, N-methylpiperidine, N-methylimidazole, N-methylpyrazole, N-methylmorpholine, N-methylhexamethylenediamine. Particular preference is given to triethylamine and N,N-diisopropylethylamine.

The reaction time is from 10 minutes to 48 hours. The reaction is carried out at temperatures between −40° C. and +150° C., preferably between −20° C. and 120° C., particularly preferably between −5° C. and 80° C. The reaction is preferably carried out under atmospheric pressure or under pressures of up to 15 bar and, if appropriate, under an atmosphere of protective gas (nitrogen, helium or argon).

After the reaction has ended, the entire reaction mixture is concentrated. The resulting compounds of the general formula (VI), for example the (4S,5R)-5-allyl-N-(2-bromo-5-fluorobenzyl)-N-but-3-en-1-yl-2,2-dimethyl-1,3-dioxolane-4-carboxamide, can be purified in a customary manner by recrystallization or column chromatography (cf. Preparation Examples).

The reaction of compounds of the general formula (IV) according to the fourth reaction step in preparation process a) is carried out by reacting the compounds of the general formula (VI), for example the (4S,5R)-5-allyl-N-(2-bromo-5-fluorobenzyl)-N-but-3-en-1-yl-2,2-dimethyl-1,3-dioxolane-4-carboxamide, under the reaction conditions of a metathesis reaction, if appropriate in the presence of a suitable catalyst and if appropriate in the presence of a diluent, to give compounds of the general formula (VII), for example (3aS,10aR)-5-(2-bromo-5-fluorobenzyl)-2,2-dimethyl-3a,5,6,7,10,10a-hexahydro-4H-[1,3]dioxolo[4,5-c]azonin-4-one.

The metathesis reaction is known from the literature and can be carried out under the reaction conditions known to be suitable for this reaction, using known catalysts (cf., for example: Van de Weghe, P. et al., Current Topics Med. Chem. 5, 1461-1472, 2005; Deiters, A. et al., Chem. Rev. (Washington, D.C., United States) 104, 2199-2238, 2004; Nakamura, I.; Yamamoto, Y., Chem. Rev. (Washington, D.C., United States) 104, 2127-2198, 2004).

By way of example and by way of preference, use is made here of ruthenium catalysts, which are also known as Grubbs catalysts of the first and second generation (for example Schmidt, B., Angew. Chem., Intern. Edition 42, 4996-4999, 2003).

However, preferred diluents for carrying out the fourth reaction step in the process a) according to the invention are chlorinated hydrocarbons, such as tetrachloroethylene, tetrachloroethane, dichloropropane, methylene chloride, dichlorobutane, chloroform, carbon tetrachloride, trichloroethane, trichloroethylene, pentachloroethane, difluorobenzene, 1,2-dichloroethane, chlorobenzene, bromobenzene, dichlorobenzene, chlorotoluene, trichlorobenzene, in particular dichloromethane.

The reaction of compounds of the general formula (VII) according to the fourth reaction step in the preparation process a) according to the invention is carried out by reacting the compounds of the general formula (VII) in the presence of suitable catalysts, for example Grubbs catalysts of the second generation.

The reaction time is from 10 minutes to 48 hours. The reaction is carried out at temperatures between −110° C. and +200° C., preferably between 0° C. and 150° C., particularly preferably between 10° C. and 100° C. With very particular preference, the reaction is carried out initially at the reflux temperature of dichloromethane and then at room temperature.

After the reaction has ended, the entire reaction mixture is concentrated. The resulting compounds of the general formula (VII), for example (3aS,10aR)-5-(2-bromo-5-fluorobenzyl)-2,2-dimethyl-3a,5,6,7,10,10a-hexahydro-4H-[1,3]dioxolo[4,5-c]azonin-4-one, can be purified in a customary manner by recrystallization or column chromatography (cf. Preparation Examples).

The reaction of compounds of the general formula (VII) according to the fifth reaction step in preparation process a) is carried out by reacting the compounds of the general formula (VII), (3aS,10aR)-5-(2-bromo-5-fluorobenzyl)-2,2-dimethyl-3a,5,6,7,10,10a-hexahydro-4H-[1,3]dioxolo-[4,5-c]azonin-4-one under the reaction conditions of a Heck reaction, if appropriate in the presence of suitable noble metal salts and if appropriate in the presence of a suitable catalyst and if appropriate in the presence of a diluent, to give compounds of the general formula (Ia), for example (3aS,13aR)-8-fluoro-2,2-dimethyl-3a,6,13,13a-tetrahydro-4H-5,11-ethano[1,3]dioxolo[4,5-d][2]benzazecin-4-one.

The Heck reaction is known from the literature and can be carried out under reaction conditions known to be suitable for this reaction, using known catalysts (cf., for example, Dounay, A. B.; Overman, L. E., Chem. Rev. 103, 2945-2963, 2003; Li, Chao-Jun., Chem. Rev. 105, 3095-3165, 2005).

For example chiral P, N ligands containing a pyridine nitrogen and a phosphor donor atom (cf. the review: Chelucci, G. et al., Tetrahedron 59, 9471-9515, 2003; Alonso, F. et al., Tetrahedron 61, 11771-11835, 2005; solid-phase synthesis: Brase, S. et al., Tetrahedron 59, 885-939, 2003).

A preferred ligand used for carrying out the fifth reaction step in the process a) according to the invention is 1,3-bis(diphenylphosphino)propane.

Additionally, for carrying out the fifth reaction step in the process a) according to the invention, use is made of suitable noble metal salts, such as, for example, palladium salts, for example palladium(II) acetate, or silver salts, for example silver carbonate.

However, preferred diluents for carrying out the fifth reaction step in the process a) according to the invention are aromatic hydrocarbons, such as benzene, toluene, chlorobenzene, bromobenzene, nitrobenzene or xylene, and in particular toluene.

The reaction time is from 10 minutes to 48 hours. The reaction is carried out at temperatures between −10° C. and +200° C., preferably between 0° C. and 180° C., particularly preferably between +10° C. and 150° C. Very particularly preferably, the reaction is carried out at the reflux temperature of toluene.

After the reaction has ended, the entire reaction mixture is concentrated. The resulting compounds of the general formula (Ia), for example (3aS,13aR)-8-fluoro-2,2-dimethyl-3a,6,13,13a-tetrahydro-4H-5,11-ethano[1,3]dioxolo[4,5-d][2]benzazecin-4-one, can be purified in a customary manner by recrystallization or column chromatography (cf. Preparation Examples).

If, to prepare the novel compounds of the general formula (I), the compound of the formula (Ia) used is, for example, (3aS,13aR)-8-fluoro-2,2-dimethyl-3a,6,13,13a-tetrahydro-4H-5,11-ethano[1,3]dioxolo[4,5-d][2]benzazecin-4-one in the presence of a suitable oxidizing agent for C═C double bonds and in the presence of a diluent, the preparation process b), which comprises three reaction steps, can be represented by reaction scheme III below:

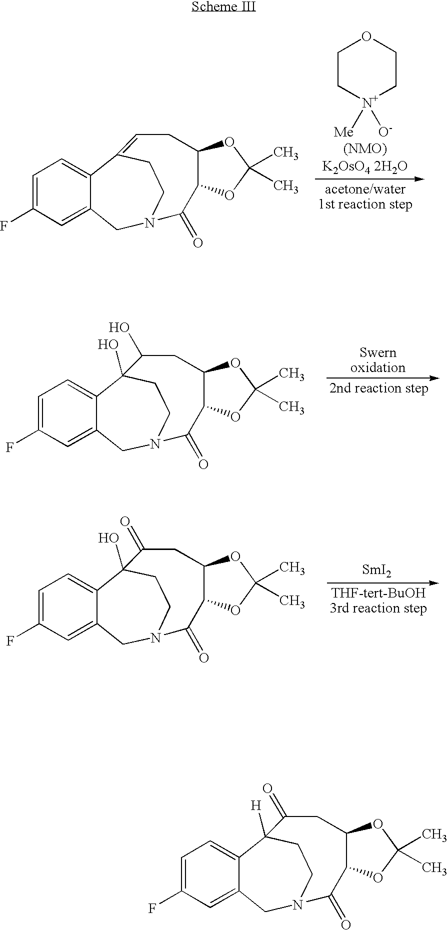

The preparation process b) is based on the total synthesis, recently published by Enders et al. (J. Org. Chem. 70, 10538-10551, 2005), of the cripowellin skeleton.

The formula (Ia) provides a general definition of the compounds required as starting materials for carrying out the process b) according to the invention.

In this formula (Ia), X and Y preferably represent those radicals which have already been mentioned in connection with the description of the novel material of the general formula (Ia) according to the invention as being preferred substituents.

The compounds of the general formula (Ia), for example (3aS,13aR)-8-fluoro-2,2-dimethyl-3a,6,13,13a-tetrahydro-4H-5,11-ethano[1,3]dioxolo[4,5-d][2]benzazecin-4-one, can be obtained by the preparation process a) according to the invention, mentioned further above.

In general, it is advantageous to carry out the preparation process b) according to the invention in the presence of diluents.

Preferred diluents for carrying out the first reaction step in the process b) according to the invention are ketones, such as acetone, acetophenone, methyl ethyl ketone or methyl butyl ketone, in particular acetone in combination with water.

The reaction of compounds of the general formula (Ia), for example (3aS,13aR)-8-fluoro-2,2-dimethyl-3a,6,13,13a-tetrahydro-4H-5,11-ethano[1,3]dioxolo[4,5-d][2]benzazecin-4-one, according to the first reaction step in the preparation process b) according to the invention is carried out by reacting the compounds of the general formula (Ia) in the presence of suitable oxidizing agents.

A large number of different oxidizing agents is known to be suitable for oxidizing alcoholic groups (cf., for example, oxidating agents in: Organic Synthesis by Oxidation with Metal Compounds; Mijs, de Jonge; Plenum Verlag: New York, 1986; Manganese Compounds as Oxidising Agents in Organic Chemistry; Arndt, Open Court Publishing Company: La Salle, Ill., 1981; The Oxidation of Organic Compounds by Permanganate Ion and Hexavalent Chromium; Lee, Open Court Publishing Company: La Salle, Ill., 1980). Accordingly, an oxidation can be carried out, for example, in the presence of permanganates, halogens, such as chlorine or bromine, metal oxides, such as manganese dioxide or ruthenium tetroxide, inter alia.

Also described in the literature are numerous different oxidizing agents specifically just for the oxidation of secondary alcohols, such as, for example, the use of acidic dichromates (cf. Chromium Oxidations in Organic Chemistry; Cainelli, Cardillo, Springer Verlag: New York, 1984; Reagents for Organic Synthesis; Fieser, Vol. 1, Wiley: New York, 1967, pp. 142-147, 1059-1064 and further volumes in this series). A solution of chromic acid and sulphuric acid in water is known as Jones reagent. Three other chromium(VI) reagents (see communication of a comparative study of Jones, Collins and Corey reagents in Warrener et al., Aust. J. Chem. (1978), 31, 1113) are also used, as is known; for example dipyridine/chromium(VI) oxide (Collins reagent; cf. Collins et al., Tetrahetron Lett., 3363, 1968), pyridinium chlorochromate (Corey reagent) and pyridinium dichromate. For acid-sensitive substrates, for example, the use of chromium(VI) oxide in hexamethylphosphoric triamide (HMPA) (Cardillo et al., Synthesis, 394, 1976), a chromium(VI) oxide/pyridine complex (Poos et al., J. Am. Chem. Soc. 75, 422, 1953) or trimethylsilyl chromates (Moiseenkov et al., J. Org. Chem. USSR 23, 1646, 1987) have also been described. Sodium hypochlorite in acetic acid has been mentioned for the oxidation of the relatively large quantity of a secondary alcohol (cf. Stevensens et al., J. Org. Chem. 45, 2030, 1980; Schneider et al., J. Org. Chem. 47, 364, 1982). If appropriate, the oxidizing agents can also be present attached to polymers (cf. review: McKillop, Young, Synthesis, 401-422, 1979). In this manner, both chromic acids and permanganates have been used as oxidizing agents. Also known are numerous phase-transfer reactions with permanganates, chromic acids (Hutchins et al., Tetrahedron Lett., 4167, 1977; Landini et al., Synthesis, 134, 1979) and ruthenium tetroxide (Morris, Kiely, J. Org. Chem. 52, 1149, 1987). Even ultrasound-induced oxidation reactions are feasible—thus, the use of potassium permanganate is mentioned (Yamwaki et al., Chem. Lett., 379, 1983).

In addition, most of the oxidizing agents which are capable of oxidizing primary alcohols to aldehydes are suitable, as they are for the corresponding oxidation of secondary alcohols. Such oxidizing agents for primary alcohols are, for example, pyridinium dichromate, tetrapropylammonium perruthenate (Pr₄N⁺ RuO₄⁻), cerium ammonium nitrate (CAN), silver carbonate on Celite (Fetizon et al., Acad. Sci., Ser. C 267, 900, 1968), Na₂Cr₂O₇ in water (Lee et al., J. Org. Chem. 35, 3589, 1970), lead tetraacetate/pyridine, benzoyl peroxide/nickel(II) bromide or dimethyl sulphoxide in the presence of oxalyl chloride (Swern oxidation), copper(II) sulphate pentahydrate in pyridine, copper(II) acetate in 70% strength acetic acid, iron chloride in water, chromium(VI) oxide in glacial acetic acid or dichromium trioxide in pyridine. The reagents which are capable of specifically oxidizing a secondary hydroxyl group, even in the presence of a primary hydroxyl group, include, for example, hydrogen peroxide/ammonium molybdate (Trost et al., Isr. J. Chem. 24, 134, 1984), sodium borate (NaBrO₃)—CAN. N-Halosuccinimides (halogen=chlorine, bromine, iodine) can be employed as oxidizing agents for hydroxyl groups, even in the presence of other oxidizable groups. The combination of N-iodosuccinimide/tetrabutylammonium iodide, for example, is suitable for oxidizing secondary alcohols in high yields (Hanessian et al., Synthesis, 394, 1981).

Further known oxidation methods also include oxidative dehydrogenation, for example in the presence of catalysts, such as silver or copper catalysts (M. Muhler in: Handbook of Heterogenous Catalysis, VCH, Weinheim, 1997). Other mild oxidative processes using platinum/carbon or palladium/carbon catalysts, which even permit the oxidation of classes of sensitive compounds, for example carbohydrates (Besson, M. et al., J. Catal. 152, 116-122, 1995) or steroids (Akihisa, T. et al., Bull. Chem. Soc. Jpn. 59, 680-685, 1986), are known. An example of an efficient commercial catalyst for the oxidation is the inorganic TS-1 catalyst (oxide titanium silicalite), which allows the catalytic oxidation of primary and secondary alcohols in aqueous hydrogen peroxide (30% w/w) (Murugawel, R. et al., Angew. Chem. Int. Ed. Engl. 36, 477-479, 1997).

Preferred oxidizing agent for carrying out the first reaction step in the process b) according to the invention are osmium compounds, in particular K₂OsO₄ in the presence of N-methylmorpoline-N-oxide (NMO).

Preferred oxidizing agent for carrying out the second reaction step in the process b) according to the invention is dimethyl sulphoxide in the presence of oxalyl chloride, i.e. the Swern oxidation.

The reaction time of the first reaction step is from 10 minutes to 48 hours. The reaction is carried out at temperatures between −10° C. and +200° C., preferably between 0° C. and 180° C., particularly preferably between +10° C. and 150° C. Very particularly preferably, the reaction is carried out at room temperature.

After the reaction has ended, the entire reaction mixture is concentrated. The resulting compounds of the general formula (I; R′═OH, A=CHOH) can be purified in a customary manner by recrystallization, vacuum distillation or column chromatography. However, alternatively, the compounds of the general formula (I; R′═OH, A=CHOH) can be used without further purification for carrying out the second reaction step (cf. Preparation Examples).

The reaction of compounds of the general formula (I; R′═OH, A=CHOH) according to the second reaction step in preparation process b) is carried out by reacting the compounds of the general formula (I; R′═OH, A=CHOH) in the presence of another oxidizing agent and in the presence of a basic reaction auxiliary in one of the diluents mentioned further above.

However, preferred diluents for carrying out the second reaction step in the process b) according to the invention are chlorinated hydrocarbons, such as tetrachloroethylene tetrachloroethane, dichloropropane, methylene chloride, dichlorobutane, chloroform, carbon tetrachloride, trichloroethane, trichloroethylene, pentachloroethane, difluorobenzene, 1,2-dichloroethane, chlorobenzene, bromobenzene, dichlorobenzene, chlorotoluene, trichlorobenzene, in particular dichloromethane.

Preferred reaction conditions used for the oxidation are the reaction conditions, known from the literature, of the Swern oxidation.

Preferred basic auxiliaries are tertiary amines, such as triethylamine, trimethylamine, tribenzylamine, triisopropylamine, tributylamine, tricyclohexylamine, triamylamine, N,N-dimethyltoluidine, N,N-dimethyl-p-aminopyridine, N-methylpyrrolidine, N-methylpiperidine, N-methylimidazole, N-methylpyrazole, N-methylmorpholine, N-methylhexamethylenediamine. Particular preference is given to triethylamine and N,N-diisopropylethylamine.

The reaction time of the second reaction step is from 10 minutes to 48 hours. The reaction is carried out at temperatures between −100° C. and +150° C., preferably between −90° C. and 100° C., particularly preferably between −80° C. and 50° C. The reaction is preferably carried out under atmospheric pressure or under pressures of up to 15 bar and, if appropriate, under an atmosphere of protective gas (nitrogen, helium or argon).

After the reaction has ended, the entire reaction mixture is concentrated. The resulting compounds of the general formula (I, R′═OH, A=CO), for example (3aS,13aR)-11-fluoro-[1-hydroxy-2,2-dimethyl-6,13,13a-tetrahydro-4H-5,11-ethano[1,3]dioxolo[4,5-d][2]benzazecin-4,12(3aH)-dione, can be purified in a customary manner by recrystallization, vacuum distillation or column chromatography (cf. Preparation Examples).

The reaction of compounds of the general formula (I; R′═OH, A=CO) according to the third reaction step in preparation process b) is carried out by reacting the compounds of the general formula (I; R′═OH, A=CO), for example (3aS,13aR)-8-fluoro-11-hydroxy-2,2-dimethyl-6,13,13a-tetrahydro-4H-5,11-ethano[1,3]dioxolo[4,5-d][2]benzazecin-4,12(3aH)-dione, in the presence of a suitable salt of a lanthanoid metal in one of the diluents mentioned further above.

Known lanthanoid metals are lanthanum, cerium, praseodymium, neodymium, promethium, samarium, europium, gadolinium, terbium, dysprosium, holmium, erbium, thulium, ytterbium and lutetium. Preferred salts of the lanthanoid metals are, for example, chlorides, bromides, iodides, acetates or carbonates. In preparation process b), according to the invention, samarium(II) iodide is used as a particularly preferred salt of a lanthanoid metal.

Preferred diluents for carrying out the third reaction step in the process b) according to the invention are mixtures of ethers, such as ethyl propyl ether, methyl tert-butyl ether, n-butyl ether, anisole, phenetol, cyclohexyl methyl ether, dimethyl ether, diethyl ether, dipropyl ether, diisopropyl ether, di-n-butyl ether, diisobutyl ether, diisoamyl ether, ethylene glycol dimethyl ether, tetrahydrofuran or dioxane. Preference is given to using a mixture of tert-butanol and tetrahydrofuran.

The reaction time of the third reaction step is from 10 minutes to 48 hours. The reaction is carried out at temperatures between −10° C. and +150° C., preferably between 0° C. and 100° C., particularly preferably between 10° C. and 50° C. The reaction is preferably carried out at room temperature. Furthermore, the reaction is carried out under atmospheric pressure or under pressures of up to 15 bar and, if appropriate, under an atmosphere of protective gas (nitrogen, helium or argon).

After the reaction has ended, the entire reaction mixture is concentrated. The resulting compounds of the general formula (I, R′═H, A=CO), for example (3aS,13aR)-8-fluoro-2,2-dimethyl-6,11,13,13a-tetrahydro-4H-5,11-ethano[1,3]dioxolo[4,5-d][2]benzazecin-4,12(3aH)-dione, can be purified in a customary manner by recrystallization, vacuum distillation or column chromatography (cf. Preparation Examples).

For preparing the compounds of the general formula (I) according to the invention in which Q represents sulphur, it is possible to use as suitable starting materials, for example, the compounds of the general formulae (Ia; Q=oxygen) and (I; Q=oxygen), in which R′ represents hydrogen or hydroxyl and A represents a carbonyl function which, if appropriate, is protected by a method known from the literature (cf. reaction scheme IV):

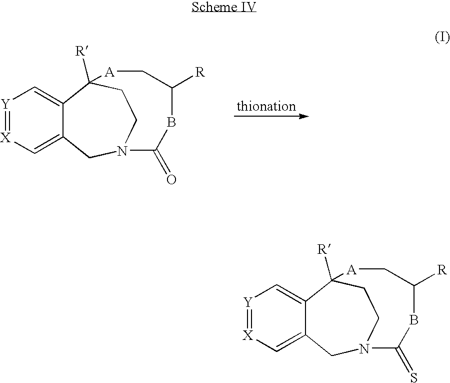

A large number of different sulphurizing agents are described in the literature, such as, for example, hydrogen sulphide (H₂S), hydrogen sulphide/hydrogen chloride (H₂S/HCl), hydrogen persulphide/hydrogen chloride (H₂S₂/HCl), di(diethylaluminium) sulphide [(Et₂Al)₂S], polymeric ethylaluminium sulphide [(EtAIS)ₙ], silicon disulphide (SiS₂), diboron trisulphide (B₂S₃), phosphorus pentachloride/dialuminium sulphide/sodium sulphate (PCl₃/Al₂S₃/NaSO₄), sodium sulphide/sulphuric acid (Na₂S/H₂SO₄), diphosphorus pentasulphide (P₂S₅), diphosphorous pentasulphide/pyridine (P₂S₅/Py), diethylthiocarbamoyl chloride, diphosphorus pentasulphide/triethylamine (P₂S₅/NEt₃), diphosphorus pentasulphide/n-butyllithium (P₂S₅/n-BuLi), diphosphorus pentasulphide/sodium bicarbonate (P₂S₅/NaHCO₃; “Scheeren reagent”, formation of Na²⁺ [P₄S₁₀O]²⁻), diphosphorus pentasulphide/methanol (P₂S₅/MeOH), SCN—CO-OEt, PSClₓ (NMe₂)₃₋ₓ (x=0-3), bis(1,5-cyclooctanediylboryl) sulphide [(9-BBN)₂S] as sulphurizing agent or as phosphorus pentasulphide replacement, 2,4-bis(methylthio)-1,3,2,4-dithiadiphosphetane 2,4-disulphide “Davy reagent methyl” (DR-Me), 2,4-bis(ethylthio)-1,3,2,4-dithiadiphosphetane 2,4-disulphide “Davy reagent ethyl” (DR-Et), 2,4-bis(para-tolylthio)-1,3,2,4-dithiadiphosphetane 2,4-disulphide “Davy reagent para-tolyl” or “Heimgartner reagent” (DR-T), 2,4-bis(4-phenoxyphenyl)-2,4-dithioxo-1,3,2,4-dithiadiphosphetane “Belleau reagent (BR)”, 2,4-bis(4-phenylthiophenyl)-2,4-dithioxo-1,3,2,4-dithiadiphosphetane, 2,4-bis(4-methoxyphenyl)-2,4-dithioxo-1,3,2,4-dithiadiphosphetane “Lawesson reagent (LR)” (cf. Davy's reagent: Heimgartneret, H. al., Helv. Chim. Acta 70, 1001, 1987; Belleau reagent: Jensen, O. E. et al., Tetrahedron 41, 5595, 1985; Lawesson reagent: Cherkasov, R. A. et al., Tetrahedron 41, 2567, 1985; diboryl sulphide: Metzner et al. In Sulphur Reagents in Organic Synthesis, B. Harcourt: London 1994, Academic Press, pp. 44-54).

Also possible are alternative reaction sequences, such as, for example, in O-alkylation with R₃⁺ BF⁴⁻ (R=methyl, ethyl) (H. Meerwein et al., Justus Liebigs Ann. Chem. (1961), 641, p. 1) and subsequent reaction of the intermediates with anhydrous NaSH(R. E. Eibeck, Inorg. Syn. (1963) 7, p. 128), the in situ formation of chloriminium salts and the subsequent reaction with tetrathiomolybdates, in particular benzyltriethylammonium tetrathiomolybdate [(PhCH₂NEt₃)₂MoS₄], or hexamethyldisilathiane (TMS₂S).

The compounds of the formulae (Ib) and (Ic) can be prepared by the processes described in WO 97/34910. In particular, they can also be prepared by the process described by Moon et al. (Organic Letters 7, 1031-1034, 2005).

The compounds of the formula (I) stabilize microtubuli. Accordingly, they can be used for treating a large number of types of cancer and other proliferative diseases. Examples of such disorders are:

carcinomas, including carcinomas of the bladder, breast, colon, kidneys, liver, lungs, ovaries, pancreas, stomach, cervix, thyroid and skin, including squamous carcinomas;
haematopoetic tumours of the lymphoid cell, including leukaemias, acute lymphocytic leukaemias, acute lymphoblastic leukaemias, B-cell lymphomas, T-cell lymphomas, Hodgkin lymphomas, non-Hodgkin lymphomas, hairy cell lymphomas and Burketts lymphomas;
haematopoetic tumours of the myeloid cell line, including acute and chronic myelogenic leukaemias and promyelocytic leukaemias;
tumours of mesenchymal origin, including fibrosarcomas and rhabdomyosarcomas;
tumours of the central and peripheral nervous system, including astrocytomas, neuroblastomas, gliomas and schwannomas;
tumours of mesenchymal origin, including fibrosarcomas, rhabdomyosarcomas and osteosarcomas; and
other tumours, including melanomas, seminomas, teratocarcinomas, neuroblastomas, gliomas, Xenoderma pigmentosum, keratocanthomas and follicular thyroid carcinomas.

If appropriate, the compounds of the formula (I) are also capable of inhibiting angiogenesis, and they can thus influence the growth of tumours. Such antiangiogenetic properties are also useful when treating other disorders responding to antiangiogenetic agents, for example certain forms of blindness associated with vascularization of the retina, arthritis, in particular inflammatory arthritis, multiple sclerosis, restenosis and psoriasis.

The compounds of the formula (I) induce or inhibit apoptosis, a physiological process resulting in the death of cells, which is critical for normal development and homeostasis. Changes of the apoptotic pathways contribute to the pathogenesis of a large number of human disorders. Accordingly, as modulators of apoptosis, the compounds can be of benefit in the treatment of a large number of disorders of man with aberrations of apoptosis, for example precancerous lesions, disorders associated with the immune response, viral infections, degenerative disorders of the musculoskeletal system and kidney disorders.

The effective amount of a compound of the formula (I) can be determined by a person of average skill in the art and includes exemplary dosage amounts for a human of about 0.05 to 200 mg/kg/day which can be administered in a single dose or in the form of individual separate doses, such as 1 to 4 times per day. Preferably, the compounds are administered in a dosage of less than 100 mg/kg/day, in a single dose or in 2 to 4 separate doses. It is evident that the specific dose and the dosage frequency for a certain patient can be varied and depend on a large number of factors, including the effectiveness of the particular compound used, the metabolic stability and the duration of action of this compound, the species, the age, the body weight, the general state of health, the sex and the diet of the patient, the mode and the time of administration, the elimination rate, the medicament combination and the severity of the particular disorder.

Thus, the present invention provides a medicament for humans which comprises at least one compound of the formula (I) and which allows the treatment of cancer and other proliferation diseases, in an amount effective in this context, and a pharmaceutically acceptable carrier or a pharmaceutically acceptable diluent. The compositions according to the invention may comprise other therapeutic agents as described below and can be formulated using, for example, customary solid or liquid carriers or diluents, such as pharmaceutical additives of a type suitable for the desired administration (for example excipients, binders, preservatives, stabilizers, flavours, etc.), using techniques which are well known in the field of pharmaceutical formulation or required by standard pharmaceutical practice.

The compounds of the formula (I) can be administered by any suitable means, for example orally, such as in the form of tablets, capsules, granules or powder, sublingually, buccally, parenterally, such as by subcutaneous, intravenous, intramuscular or intrasternal injection or infusion techniques (for example as sterile, injectable, aqueous or non-aqueous solutions or suspensions), nasally, such as by means of an inhalation spray; topically, such as in the form of a cream or ointment, or rectally, such as in the form of suppositories, in dosage unit formulations comprising non-toxic pharmaceutically acceptable carriers or diluents. The compounds of the formula (I) can be administered, for example, in a form suitable for immediate release or delayed release. Immediate release or delayed release can be achieved by using suitable medicaments comprising the compounds of the formula (I) or, in particular in the case of a delayed release, by using devices such as subcutaneous implants or osmotic pumps. The compounds of the formula (I) can also be administered in liposomal form. The active substance can be used, for example, in a composition such as a tablet, a capsule, a solution or suspension comprising about 5 to about 500 mg per unit dose of a compound or a mixture of compounds of the formula or in a topical form (0.01 to 5% by weight of the compound of the formula (I), one to five treatments per day). It can be mixed in a customary manner with a physiologically acceptable carrier, excipient, binder, preservative, stabilizer, flavour, etc., or with a topical carrier. The compounds of the formula (I) can also be formulated in compositions, such as sterile solutions or suspensions, for parenteral administration. About 0.1 to 500 mg of a compound of the formula (I) can be mixed with a physiologically acceptable carrier, excipient, binder, preservative, stabilizer, etc., in a unit dosage form, as required by standard pharmaceutical practice. The amount of active substance in these compositions or preparations is preferably such that a suitable dosage in the stated range is obtained.

Exemplary compositions for oral administration include suspensions which may comprise, for example, microcrystalline cellulose to increase the bulk, alginic acid or sodium alginate as suspending agent, methylcellulose as viscosity-increasing agent, and sweeteners or flavours such as those known in the art, and tablets with immediate release which may, for example, comprise microcrystalline cellulose, dicalcium phosphate, starch, magnesium stearate and/or lactose and/or other excipients, binders, extenders, peptizers, diluents and glidants, such as those known in the art. Formed tablets, pressed tablets or freeze-dried tablets are exemplary forms which may be used. Exemplary compositions include those which formulate the compounds of the formula (I) with rapidly soluble solvents, such as mannitol, lactose, sucrose and/or cyclodextrins. Such formulations may also comprise excipients of high molecular weight, such as celluloses (Avicel) or polyethylene glycols (PEG). Such formulations may also contain an excipient to support adhesion to the mucosa, such as hydroxypropylcellulose (HPC), hydroxypropylmethylcellulose (HPMC), sodium carboxymethylcellulose (SCMC), maleic anhydride copolymers (for example Gantrez) and agents for controlling the release, such as polyacrylate copolymer (for example Carbopol 934). Lubricants, glidants, flavours, colorants and stabilizers may also be added, to facilitate preparation and use.

Exemplary compositions for nasal aerosol and inhalation administration include solutions in physiological saline comprising, for example, benzyl alcohol and other suitable preservatives, absorption enhancers to increase the bioavailability and/or other solubilizers and dispersants, such as those known in the art.

Exemplary compositions for parenteral administration include injectable solutions or suspensions which may comprise, for example, suitable non-toxic parenterally acceptable diluents or solvents, such as Cremophor, mannitol, 1,3-butanediol, water, Ringer solution, an isotonic sodium chloride solution or other suitable dispersants; or wetting agents and suspension agents, including synthetic mono- or diglycerides, and fatty acids, including oleic acids.

Exemplary compositions for rectal administration include suppositories which may contain, for example, a suitable non-irritating excipient, such as cocoa butter, synthetic glyceride esters or polyethylene glycols, which are solid at normal temperature but liquefy and/or dissolve in the rectal cavity, releasing the medicament.

Exemplary compositions for topical administration include a topical carrier, such as Plastibase (mineral oil gelated with polyethylene). The compounds of the formula (I) can be administered topically to treat the plaques associated with psoriasis, for example, and can be formulated as such as a cream or ointment.

The compounds of the formula (I) can be administered either on their own or in combination with other anticancer and cytotoxic agents and treatments suitable for managing cancer or other proliferation disorders. Particularly useful are anticancer and cytotoxic medicament combinations in which the second chosen medicament acts in a different manner or during a different phase of the cell cycle, for example the S phase, than the present compounds of the formula (I) which are active during the G2-M phase. Examples of classes of anticancer and cytotoxic agents include alkylating agents, such as nitrogen mustard, alkylsulphonates, nitrosoureas, ethyleneimines and triazenes; antimetabolites, such as folate antagonists, purine analogues and pyrimidine analogues; antibiotics, such as anthracyclines, bleomycins, mitomycin, dactinomycin and plicamycin; enzymes, such as L-aspartase; farnesyl protein transferase inhibitors; hormonal agents, such as glucocorticoids, oestrogens/anti-oestrogens, androgens/antiandrogens, progestins and luteinizing hormone-releasing hormone antagonists, octreotide acetate; microtubulus-destroying agents, such as ectein-ascidins or their analogues and derivatives; microtubulus-stabilizing agents, such as paclitaxel (Taxol), docetaxel (Taxotere) and epothilones A-F or their analogues or derivatives; plant-derived products, such as vinca alkaloids, epipodophyllotoxins, taxanes; and topoisomerase inhibitors; prenyl protein transferase inhibitors; and various agents, such as hydroxyurea, procarbazine, mitotane, hexamethylmelamine, platinum coordination complexes, such as cisplatin and carboplatin; and other agents used as anticancer and cytotoxic agents, such as agents which modify the biological reaction, growth factors, immunomodulators and monoclonal antibodies. The compounds of the formula (I) can also be used in combination with radiotherapy.

Representative examples of these classes of anticancer and cytotoxic agents include mechlorethamine hydrochloride, cyclophosphamid, chlorambucil, melphalan, ifosfamid, busulfan, carmustine, lomustine, semustine, streptozocin, thiotepa, dacarbazin, methotrexate, thioguanine, mercaptopurine, fludarabine, pentastatin, cladribin, cytarabine, fluorouracil, doxorubicin hydrochloride, daunorubicin, idarubicin, bleomycin sulphate, mitomycin C, actinomycin D, safracins, saframycins, quinocarcins, discodermolides, vincristine, vinblastine, vinorelbine tartrate, etoposide, teniposide, paclitaxel, tamoxifen, estramustine, estramustine phosphate sodium, flutamide, buserelin, leuprolide, pteridines, diyneses, levamisole, aflacon, interferon, interleukins, aldesleukin, filgrastim, sargramostim, rituximab, BCG, tretinoin, irinotecan hydrochloride, betamethasone, gemcitabine hydrochloride, altretamine and topoteca and all analogues or derivatives thereof.

Preferred members of these classes include paclitaxel, cisplatin, carboplatin, doxorubicin, caminomycin, daunorubicin, aminopterin, methotrexate, methopterin, mitomycin C, ectein-ascidin 743, porfiromycin, 5-fluorouracil, mercaptopurine, gemcitabine, cytosine arabinoside, podophyllotoxin or podophyllotoxin derivatives, such as etoposide, etoposide phosphate or teniposide, melphalan, vinblastine, vincristine, leurosidine, vindesine and leurosine.

The combinations of the present invention can also be formulated or administered together with other therapeutic agents selected by virtue of their particular usefulness in the administration of therapies associated with the disorders listed above. The compounds of the formula (I) can be formulated, for example, with agents such as antiemetics and H1 and H2 antihistamines, to prevent nausea, hypersensitivity and stomach irritations.

When used in combination with the compounds of the formula (I), the therapeutic agents listed above can be administered in the amounts stated in the Physicians' Desk Reference (PDR) or otherwise determined by a person of average skill.

### PREPARATION EXAMPLES

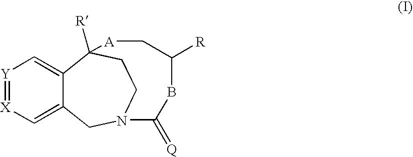

### Example I-1 (3aS,13aR)-11-Hydroxy-2,2,9-trimethyl-6,11,13,13a-tetrahydro-4H-5,11-ethano[1,3]dioxolo[4,5-d][2]benzazecin-4,12(3aH)-one

(X═CH, Y═CMe, A=C═O, R—B═O-CMe₂-O—HC, R′═OH, Q=O)

4 mg of K₂OsO₄·2H₂O and 67 mg of N-methylmorpholine N-oxide are added to a solution of 56 mg of (3aS,13aR)-2,2,9-trimethyl-3a,6,13,13a-tetrahydro-4H-5,11-ethano[1,3]dioxolo[4,5-d][2]-benzazecin-4-one in a mixture of 1.25 ml of acetone and 0.85 ml of water. After 3 hours of stirring at 20° C., 50 mg of Na₂SO₃ are added, and the reaction mixture is stirred for 5 minutes and extracted with dichloromethane. The combined organic phases are dried over magnesium sulphate and concentrated under reduced pressure. The residue obtained is used as such for the next step. 0.07 ml of dimethyl sulphoxide is added to a solution, cooled to −78° C., of 0.04 ml of oxalyl chloride in 1.5 ml of dichloromethane. After 15 minutes, the residue described above, dissolved in 1 ml of dichloromethane, is slowly added dropwise. The mixture is stirred at −78° C. for 30 min, 0.25 ml of triethylamine is added and the mixture is stirred at −78° C. for another 10 min. After warming to 20° C., the mixture is partitioned between water and dichloromethane, the combined organic phases are dried over sodium sulphate and concentrated under reduced pressure and the residue is purified by column chromatography on silica gel. This gives 25 mg of (3aS,13aR)-11-hydroxy-2,2,9-trimethyl-6,11,13,13a-tetrahydro-4H-5,11-ethano[1,3]dioxolo[4,5-d][2]benzazecine-4,12(3aH)-dione as an oil.

¹H-NMR (CD₃CN): δ [ppm]=1.47 (s, 3H), 1.48 (s, 3H), 2.03-2.09 (m, 1H), 2.34 (s, 3H), 2.73 (dd, 1H), 3.00-3.09 (m, 1H), 3.14-3.23 (m, 1H), 3.45 (t, 1H), 3.81-3.89 (m, 1H), 3.99-4.07 (m, 1H), 4.09 (d, 1H), 4.24 (s, 1H), 4.62 (d, 1H), 4.73 (d, 1H), 7.10 (m, 2H), 7.45 (s, 1H).

logP (pH 2.7): 1.61.

Examples I-2 to I-4 are prepared analogously to Example I-1.

### Example I-2 (3aS,13aR)-8-Fluoro-11-hydroxy-2,2-dimethyl-6,11,13,13a-tetrahydro-4H-5,11-ethano[1,3]dioxolo[4,5-d][2]benzazecine-4,12(3aH)-dione

(X═CF, Y═CH, A=C═O, R—B═O-Cme₂-O—HC, R′═OH, Q=O)

Starting with 37 mg of (3aS,13aR)-8-fluoro-2,2-dimethyl-3a,6,13,13a-tetrahydro-4H-5,11-ethano[1,3]dioxolo[4,5-d][2]benzazecin-4-one, 7 mg of (3aS,13aR)-8-fluoro-2,2-dimethyl-3a,6,13,13a-tetrahydro-4H-5,11-ethano[1,3]dioxolo[4,5-d][2]benzazecin-4-one are obtained as an oil.

¹H-NMR (CD₃CN): δ [ppm]=1.47 (s, 3H), 1.48 (s, 3H), 2.05-2.11 (m, 1H), 2.73 (dd, 1H), 3.02-3.12 (m, 1H), 3.15-3.23 (m, 1H), 3.44 (t, 1H), 3.86-3.93 (m, 1H), 3.99-4.05 (m, 1H), 4.09 (d, 1H), 4.36 (s, 1H), 4.62 (d, 1H), 4.80 (d, 1H), 6.98 (m, 1H), 7.04 (m, 1H), 7.65 (dd, 1H).

logP (pH 2.7): 1.49.

### Example I-3 12-Hydroxy-9,10,12-trihydro-5H-6,12-ethano[1,3]dioxolo[4,5-k][2]benzazecine-7,11(8H)-dione

(X—Y═C—O—CH₂—O—C, A=C═O, R′═OH, R═H, B═CH₂, Q=O)

Starting with of 24 mg 5,8,9,10-tetrahydro-7H-6,12-ethano[1,3]dioxolo[4,5-k][2]benzazecin-7-one, 9 mg of 12-hydroxy-9,10,12-trihydro-5H-6,12-ethano[1,3]dioxolo[4,5-k][2]benzazecine-7,11(8H)-dione are obtained as an oil.

¹H-NMR (CDCl₃): δ [ppm]=2.10-2.23 (m, 4H), 2.32-2.37 (m, 2H), 2.55-2.58 (m, 1H), 2.65-2.71 (m, 1H), 3.15 (s, 1H), 3.35 (ddd, 1H), 3.70-3.78 (m, 1H), 4.00 (d, 1H), 5.27 (d, 1H), 5.95 (d, 1H), 5.97 (d, 1H), 6.79 (s, 1H), 7.16 (s, 1H).

logP (pH 2.7): 0.99.

### Example I-4 (3aS,14aR)-12-hydroxy-2,2-dimethyl-6,12,14,14a-tetrahydro-4H-5,12-ethanobis[1,3]dioxolo[4,5-d:4′,5′-k][2]benzazecine-4,13(3aH)-dione

(X—Y═C—O—CH₂—O—C, A=C═O, R—B═O-CMe₂-O—HC, R′═OH, Q=O) (cf. Enders, D. et al., Angew. Chem. Int. Ed. 44, 3766-3769, 2005

¹H-NMR (CD₃CN): δ [ppm]=1.47 (s, 3H), 1.47 (s, 3H), 2.04-2.10 (m, 1H), 2.71 (dd, 1H), 2.98-3.07 (m, 1H), 3.15-3.24 (m, 1H), 3.42 (t, 1H), 3.81-3.88 (m, 1H), 3.97-4.04 (m, 1H), 3.99 (d, 1H), 4.28 (s, 1H), 4.60 (d, 1H), 4.67 (d, 1H), 5.94 (d, 1H), 5.98 (d, 1H), 6.71 (s, 1H), 7.14 (s, 1H).

logP (pH 2.7): 1.38.

### Example I-5 (3aS,13aR)-2,2,9-Trimethyl-6,11,13,13a-tetrahydro-4H-5,11-ethanol[1,3]dioxolo[4,5-d][2]benzazecine-4,12(3aH)-dione

(X═CH, Y═CMe, A=C═O, R—B═O-CMe₂-O—HC, R′═H, Q=O)

25 mg of (3aS,13aR)-11-hydroxy-2,2,9-trimethyl-6,11,13,13a-tetrahydro-4H-5,11-ethano[1,3]-dioxolo[4,5-d][2]benzazecine-4,12(3aH)-dione are dissolved in 1 ml of tetrahydrofuran, and 21 μl of tert-butanol are added. Samarium(II) iodide solution (0.1N in tetrahydrofuran) is added until the colour of the solution remains green for at least 1 minute. After 16 hours of stirring at 20° C., the mixture is partitioned between diethyl ether and water, the combined organic phases are dried over sodium sulphate and concentrated under reduced pressure and the residue is purified by column chromatography on silica gel. This gives 14 mg of (3aS,13aR)-2,2,9-trimethyl-6,11,13,13a-tetrahydro-4H-5,11-ethano[1,3]dioxolo[4,5-d][2]benzazecine-4,12(3aH)-dione as an oil.

¹H-NMR (CDCl₃): δ [ppm]=1.52 (s, 3H), 1.58 (s, 3H), 2.31 (s, 3H), 2.36-2.43 (m, 1H), 2.83 (dd, 1H), 2.97-3.00 (m, 1H), 3.24-3.31 (m, 1H), 3.28 (t, 1H), 3.46 (dd, 1H), 3.85 (dd, 1H), 4.12 (d, 1H), 4.26 (ddd, 1H), 4.47 (d, 1H), 5.08 (d, 1H), 6.81 (s, 1H), 7.08 (s, 2H).

logP (pH 2.7): 1.92.

Examples I-6 and I-7 are prepared analogously to Example I-5.

### Example I-6 (3aS,13aR)-8-Fluoro-2,2-dimethyl-6,11,13,13a-tetrahydro-4H-5,11-ethano[1,3]dioxolo[4,5-d][2]benzazecine-4,12(3aH)-dione

(X═CF, Y═CH, A=C═O, R—B═O-Cme₂-O—HC, R′═H, Q=O)

Starting with 9 mg of (3aS,13aR)-8-fluoro-11-hydroxy-2,2-dimethyl-6,11,13,13a-tetrahydro-4H-5,11-ethano[1,3]dioxolo[4,5-d][2]benzazecine-4,12(3aH)-dione, 1 mg of (3aS,13aR)-8-fluoro-2,2-dimethyl-6,11,13,13a-tetrahydro-4H-5,11-ethano[1,3]dioxolo[4,5-d][2]benzazecine-4,12(3aH)-dione is obtained as an oil.

¹H-NMR (CDCl₃): δ [ppm]=1.52 (s, 3H), 1.59 (s, 3H), 2.40-2.44 (m, 1H), 2.84 (dd, 1H), 2.96-3.02 (m, 1H), 3.25-3.30 (m, 1H), 3.28 (t, 1H), 3.54 (dd, 1H), 3.87 (dd, 1H), 4.11 (d, 1H), 4.24 (ddd, 1H), 4.46 (d, 1H), 5.13 (d, 1H), 6.91-6.93 (m, 2H), 6.97 (dd, 1H).

logP (pH 2.7): 1.69.

### Example I-7 9,10,12-Trihydro-5H-6,12-ethano[1,3]dioxolo[4,5-k][2]benzazecine-7,11(8H)-dione

(X—Y═C—O—CH₂—O—C, A=C═O, R═H, B═CH₂, R′═H, Q=O)

Starting with 7 mg of 12-hydroxy-9,10,12-trihydro-5H-6,12-ethano[1,3]dioxolo[4,5-k][2]-benzazecine-7,11(8H)-dione, 3 mg of 9,10,12-trihydro-5H-6,12-ethano[1,3]dioxolo[4,5-k][2]-benzazecine-7,11(8H)-dione are obtained as an oil (cf. also Moon, B., et al., Org. Lett. (2005), 7, 1031-1034).

¹H-NMR (CDCl₃): δ [ppm]=2.04-2.24 (m, 4H), 2.31-2.41 (m, 2H), 2.66-2.71 (m, 1H), 2.90-2.95 (m, 1H), 3.34 (ddd, 1H), 3.54-3.55 (m, 1H), 3.88 (d, 1H), 3.90 (ddd, 1H), 5.33 (d, 1H), 5.93 (d, 1H), 5.96 (d, 1H), 6.47 (s, 1H), 6.75 (s, 1H).

logP (pH 2.7): 1.30.

### Determination of the X-Ray Structure of Example I-7:

Crystals suitable for determining the x-ray structure were obtained by crystallization from ethyl acetate. The lattice constants and the reflex intensities were determined using a Bruker-Nonius diffractometer. The structure was resolved using direct methods (program system SHELXTL version 6.10). Using the program SHELXTL version 6.10 against F², the structure was refined.

### Crystal Data and Refined Structure:

| | | |
|---|---|---|
| Empirical formula | C₁₆H₁₇NO₄ | |
| Molar mass | 287.31 | |
| Temperature | 90 K | |
| Wavelength | 1.54178 Å | |
| Crystal system | monoclinic | |
| Space group | P21/c | |
| Cell dimensions | a = 11.9973(8) Å | α= 90 °. |
| | b = 9.6462(5) Å | β= 104.711 (5)°. |
| | c = 11.5904(7) Å | γ= 90°. |

| | |
|---|---|
| Cell volume | 1297.37(13) Å³ |
| Formula units per cell Z | 4 |
| Density (calculated) | 1.471 Mg/m³ |
| Absorption coefficient | 0.875 mm⁻¹ |
| F(000) | 608 |
| Crystal dimensions | 0.20 × 0.10 × 0.08 mm³ |
| Theta range for data | 3.81 to 71.60°. |
| collection | |
| Index range | −13 ≦ h ≦ 14, −11 ≦ k ≦ 11, −10 ≦ 1 ≦ 14 |
| Measured reflexes | 10713 |
| Independent reflexes | 2319 [R(int) = 0.0467] |
| Completeness for | 91.7% |
| theta = 71.60° | |
| Absorption correction | SADABS (Bruker-AXS) |
| Refinement method | full matrix smallest squares on F² |
| Data/restraints/ | 2319/0/258 |
| parameters | |
| Goodness-of-fit on F2 | 1.047 |
| Final R indices | R1 = 0.0458, wR2 = 0.1223 |
| [I > 2sigma(I)] | |
| R values (all data) | R1 = 0.0510, wR2 =0.1267 |
| Largest maximum and | 0.313 and −0.305 e.Å⁻³ |
| minimum | |

### Example I-8 (3aS,14aR)-2,2-Dimethyl-6,12,14,14a-tetrahydro-4H-5,12-ethanobis[1,3]dioxolo[4,5-d:4′,5′-k][2]benzazecine-4,13(3aH)-dione

(X—Y═C—O—CH₂—O—C, A=C═O, R—B═O-CMe₂-O—HC, R′═H, Q=O)
(cf. Enders, D. et al., Angew. Chem. Int. Ed. 44, 3766-3769, 2005)

¹H-NMR (CD₃CN): δ [ppm]=1.46 (s, 3H), 1.46 (s, 3H), 2.26-2.32 (m, 1H), 2.62 (dd, 1H), 2.96-3.02 (m, 1H), 3.18-3.23 (m, 1H), 3.28 (t, 1H), 3.51 (dd, 1H), 3.91 (dd, 1H), 3.95 (d, 1H), 3.97 (ddd, 1H), 4.55 (d, 1H), 4.85 (d, 1H), 5.92 (d, 1H), 5.95 (d, 1H), 6.54 (s, 1H), 6.70 (s, 1H).

logP (pH 2.7): 1.60.

### Example I-9 (4S,5R)-4,5-Dihydroxy-10-methyl-5,6,8-trihydro-1H-2,8-ethano-2-benzazecine-3,7(4H)-dione

(X═CH, X═CMe, A=C═O, R═OH, B═CH—OH, R′═H, Q=O)

11 mg of (3aS,13aR)-2,2,9-trimethyl-6,11,13,13a-tetrahydro-4H-5,11-ethano[1,3]dioxolo[4,5-d][2]-benzazecine-4,12(3aH)-dione are taken up in 1.5 ml of water, 25 mg of Dowex 50 are added and the mixture is stirred for 4.5 hours. The water is removed by freeze-drying and the residue is taken up in chloroform and filtered. Concentration of the filtrate under reduced pressure gives 3 mg of (4S,5R)-4,5-dihydroxy-10-methyl-5,6,8-trihydro-1H-2,8-ethano-2-benzazecine-3,7(4H)-dione as an oil.

¹H-NMR (CDCl₃): δ [ppm]=2.31 (s, 3H), 2.42-2.44 (m, 1H), 2.66 (dd, 1H), 2.95-3.00 (m, 1H), 3.00-3.03 (m, 1H), 3.28 (br. s, 1H), 3.39-3.45 (m, 2H), 3.52 (t, 1H), 3.92-3.94 (m, 1H), 4.04-4.08 (m, 1H), 4.05 (d, 1H), 4.36 (t, 1H), 5.42 (d, 1H), 6.82 (s, 1H), 7.10 (m, 2H).

logP (pH 2.7): 1.33.

### Example I-10 (3aS,14aR)-2,2-Dimethyl-6,12,14,14a-tetrahydro-4H-5,12-ethanobis[1,3]dioxolo[4,5-d:4′,5′-k][2]benzazecine-4,13(3aH)-dione

(X—Y═C—O—CH₂—O—C, A=C═O, R═OH, B═CH—OH, R′═H, Q=O)
(cf. Enders, D. et al., Angew. Chem. Int. Ed. 44, 3766-3769, 2005)

¹H-NMR (CDCl₃): δ [ppm]=2.42-2.48 (m, 1H), 2.66 (dd, 1H), 2.92-3.00 (m, 1H), 3.00-3.03 (m, 1H), 3.24 (br. s, 1H), 3.38-3.42 (m, 2H), 3.45 (t, 1H), 3.90-3.93 (m, 1H), 3.97 (d, 1H), 4.05-4.11 (m, 1H), 4.36 (t, 1H), 5.36 (dd, 1H), 5.93 (d, 1H), 5.97 (d, 1H), 6.48 (s, 1H), 6.67 (s, 1H).

logP (pH 2.7): 0.89.

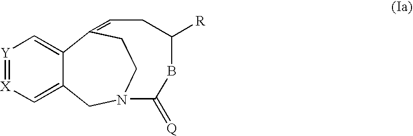

### Example Ia-1 (3aS,13aR)-2,2,9-Trimethyl-3a,6,13,13a-tetrahydro-4H-5,11-ethano[1,3]dioxolo[4,5-d][2]benzazecin-4-one

(X═CH, Y═CMe, R—B═O-Cme₂-O—HC, Q=O)

Under argon, 9 ml of toluene are added to 250 mg of (3aS,10aR)-5-(2-bromo-4-methylbenzyl)-2,2-dimethyl-3a,5,6,7,10,10a-hexahydro-4H-[1,3]dioxolo[4,5-c]azonin-4-one, 21 mg of palladium(II) acetate, 524 mg of silver carbonate and 52 mg of 1,3-bis(diphenylphosphino)propane, and the mixture is heated under reflux for 4 hours. The entire mixture is filtered through silica gel and the filter cake is washed with ethyl acetate. The filtrate is concentrated under reduced pressure and the residue is purified by column chromatography on silica gel. This gives 83 mg of (3aS,13aR)-2,2,9-trimethyl-3a,6,13,13a-tetrahydro-4H-5,11-ethano[1,3]dioxolo[4,5-d][2]benzazecin-4-one as an oil.

logP (pH 2.7): 2.62.

Examples Ia-2 to Ia-6 are prepared analogously to Example Ia-1.

### Example Ia-2 (3aS,13aR)-8-Fluoro-2,2-dimethyl-3a,6,13,13a-tetrahydro-4H-5,11-ethano[1,3]dioxolo[4,5-d][2]benzazecin-4-one

(X CF, Y═CH, R—B═O-Cme₂-O—HC, Q=O)

Starting with 108 mg of (3aS,10aR)-5-(2-bromo-5-fluorobenzyl)-2,2-dimethyl-3a,5,6,7,10,10a-hexahydro-4H-[1,3]dioxolo[4,5-c]azonin-4-one, 46 mg of (3aS,13aR)-8-fluoro-2,2-dimethyl-3a,6,13,13a-tetrahydro-4H-5,11-ethano[1,3]dioxolo[4,5-d][2]benzazecin-4-one as an oil.

logP (pH 2.7): 2.39.

### Example Ia-3 (3aS,13aR)-2,2-Dimethyl-3a,6,13,13a-tetrahydro-4H-5,11-ethano[1,3]dioxolo[4,5-c]pyrido[3,4-h]azecin-4-one

(X═CH, Y═N, R—B═O-CMe₂-O—HC, Q=O)

Starting with 169 mg of (3aS,10aR)-5-[(3-bromopyridin-4-yl)methyl]-2,2-dimethyl-3a,5,6,7,10,10a-hexahydro-4H-[1,3]dioxolo[4,5-c]azonin-4-one, 120 mg of a crude product comprising about 1% of (3aS,13aR)-2,2-dimethyl-3a,6,13,13a-tetrahydro-4H-5,11-ethano[1,3]-dioxolo[4,5-c]pyrido[3,4-h]azecin-4-one are obtained.

logP (pH 2.7): 0.49.

### Example Ia-4 10,11-Dimethoxy-1,4,5,6-tetrahydro-3H-2,8-ethano-2-benzazecin-3-one

(X, Y═C-OMe, R═H, B═CH₂, Q=O)

Starting with 200 mg of 1-(2-bromo-4,5-dimethoxybenzyl)-1,3,4,5,8,9-hexahydro-2H-azonin-2-one, 5 mg of 10,11-dimethoxy-1,4,5,6-tetrahydro-3H-2,8-ethano-2-benzazecin-3-one are obtained as an oil.

¹H-NMR (CD₃CN): δ [ppm]=1.68-1.79 (m, 2H), 1.88-2.05 (m, 3H), 2.10-2.15 (m, 1H), 2.46 (m, 1H), 2.64 (m, 1H), 2.95 (m, 1H), 3.74 (s, 3H), 3.77 (s, 3H), 4.33 (d, 1H), 4.35 (m, 1H), 4.86 (d, 1H), 5.19 (dd, 1H), 6.42 (s, 1H), 6.69 (s, 1H).

logP (pH 2.7): 1.82.

### Example Ia-5 5,8,9,10-Tetrahydro-7H-6,12-ethano[1,3]dioxolo[4,5-k][2]benzazecin-7-one

(X—Y═C—O—CH₂—O—C, R═H, B═CH₂, Q=O)

Starting with 300 Mg of 1-[(6-Bromo-1,3-Benzodioxol-5-yl)Methyl]-1,3,4,5,8,9-Hexahydro-2H-azonin-2-one, 37 mg of 5,8,9,10-tetrahydro-7H-6,12-ethano[1,3]dioxolo[4,5-k][2]benzazecin-7-one are obtained as an oil.

logP (pH 2.7): 2.01.

### Example Ia-6 (3aS,14aR)-2,2-Dimethyl-3a,6,14,14a-tetrahydro-4H-5,12-ethanobis[1,3]dioxolo[4,5-d:4′,5′-k][2]benzazecin-4-one

(X—Y═C—O—CH₂—O—C, R—B═O-CMe₂-O—HC, Q=O) (VRF 3303)
(cf. Enders, D. et al., Angew. Chem. Int. Ed. 44, 3766-3769, 2005)

logP (pH 2.7): 2.19.

### Preparation of the Starting Materials

### Compounds of the General Formula (VI)

### VI-1

### (4S,5R)-5-Allyl-N-(2-bromo-4-methylbenzyl)-N-but-3-en-1-yl-2,2-dimethyl-1,3-dioxolane-4-carboxamide

1.1 g of molecular sieve 4 Å and 234 mg of 3-buten-1-amine are added to a solution of 545 mg of 2-bromo-4-methylbenzaldehyde in 6 ml of dichloromethane. After 16 h of stirring at 20° C., the molecular sieve is filtered off and the solvent is removed under reduced pressure. The residue is taken up in methanol, and sodium borohydride is added a little at a time. After the evolution of gas has ceased, stirring is continued for another hour. The mixture is concentrated under reduced pressure and the residue is partitioned between water and dichloromethane. The organic phase is dried over sodium sulphate and concentrated under reduced pressure. At 0° C., the residue and 915 mg of 1-ethyl-2-fluoropyridinium tetrafluoroborate are added to a solution of 400 mg of (4S,5R)-5-allyl-2,2-dimethyl-1,3-dioxolane-4-carboxylic acid in 12 ml of dichloromethane. 1.2 ml of N,N-diisopropylethylamine are then added dropwise. After 16 hours of stirring at 20° C., the mixture is concentrated under reduced pressure and the residue is purified by column chromatography on silica gel. This gives 577 mg of (4S,5R)-5-allyl-N-(2-bromo-4-methylbenzyl)-N-but-3-en-1-yl-2,2-dimethyl-1,3-dioxolane-4-carboxamide as an oil.

The compounds of the general formula (VI) listed in Table 2 below can be prepared analogously.

TABLE 2

| | |
|---|---|
| | (VI) |
| 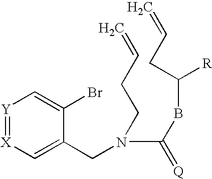 | |

| Ex. No. | X | Y | 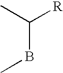 | Q | Physical data^{a)} |
|---|---|---|---|---|---|
| VI-1 | CH | CMe | 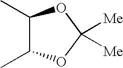 | O | 5.27 |
| VI-2 | CF | CH | 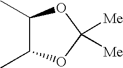 | O | 4.83 |
| VI-3 | CH | N | 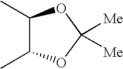 | O | 3.50 |
| VI-4 | COMe | CMe | 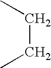 | O | 3.80 |

| | | | | |
|---|---|---|---|---|
| VI-5 | C—OCH₂O—C | 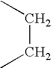 | O | 4.09 |
| VI-6* | C—OCH₂O—C | 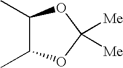 | O | 4.60 |

^{a)}logP values: the determination is carried out by HPLC on a reversed-phase column (C 18) at pH 2.7 using the mobile phases 0.1% aqueous formic acid and acetonitrile (comprimising 0.1% formic acid); linear gradient from 10% acetonitrile to 95% acetonitrile. Calibration is carried out using straight-chain alkan-2-ones (with 3 to 16 carbon atoms) having known logP values.
*cf. Enders, D. et al., Angew. Chem. Int. Ed. 44, 3766-3769, 2005

### Compounds of the General Formula (VII)

### VII-1

### (3aS,10aR)-5-(2-Bromo-4-methylbenzyl)-2,2-dimethyl-3a,5,6,7,10,10a-hexahydro-4H-[1,3]dioxolo[4,5-c]azonin-4-one

474 mg of (4S,5R)-5-allyl-N-(2-bromo-4-methylbenzyl)-N-but-3-en-1-yl-2,2-dimethyl-1,3-dioxolane-4-carboxamide are dissolved in 1.5 l of dichloromethane. The reaction vessel, together with the solution, is evacuated and flushed with argon, four times in total. The solution is heated to reflux, and over a period of 2 h, a solution of 95 mg of Grubbs II catalyst in 10 ml of dichloromethane is added a little at a time. After the addition has ended, the mixture is boiled under reflux for another hour and then cooled to 20° C. 400 μl of DMSO are added, and the mixture is stirred at 20° C. for 16 h. The mixture is concentrated under reduced pressure and the residue is purified by column chromatography on silica gel. This gives 297 mg of (3aS,10aR)-5-(2-bromo-4-methylbenzyl)-2,2-dimethyl-3a,5,6,7,10,10a-hexahydro-4H-[1,3]dioxolo[4,5-c]azonin-4-one as an oil.

The compounds of the general formula (VII) listed in Table 3 below can be prepared analogously.

TABLE 3

| | |
|---|---|
| | (VII) |
| 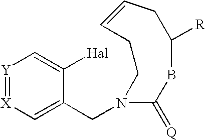 | |

| Ex. No. | X | Y | 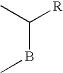 | Q | Physical data^{a)} |
|---|---|---|---|---|---|
| VII-1 | CH | CMe | 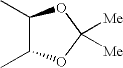 | O | 1.40 (s, 3 H), 1.44 (s, 3 H), 2.30 (s, 3 H), 2.27-2.32 (m, 1 H), 2.45-2.57 (m, 3 H), 3.23-3.30 (m, 1 H), 3.84- 3.89 (m, 2 H), 3.98 (d, 1 H), 4.57 (d, 1 H), 4.93 (d, 1 H), 5.57-5.62 (m, 1 H), 5.73-5.76 (m, 1 H), 7.11 (d, 1 H), 7.16(dd, 1 H), 7.44 (d, 1 H). logP (pH 2.7): 3.41. |
| VII-2 | CF | CH | 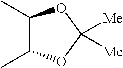 | O | 1.42 (s, 3 H), 1.44 (s, 3 H), 2.32- 2.35 (m, 1 H), 2.47-2.64 (m, 3 H), 3.26-3.33 (m, 1 H), 3.83-3.90 (m, 1 H), 3.93-4.00 (m, 1 H), 3.98 (d, 1 H), 4.60 (d, 1 H), 4.91 (d, 1 H), 5.59-5.64 (m, 1 H), 5.75-5.77 (m, 1 H), 6.96-7.01 (m, 2 H), 7.60 (dd, 1 H). logP (pH 2.7): 3.09. |
| VII-3 | CH | N | 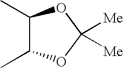 | O | 1.42 (s, 3 H), 1.45 (s, 3 H), 2.35- 2.37 (m, 1 H), 2.47-2.58 (m, 3 H), 3.26-3.34 (m, 1 H), 3.83-3.89 (m, 1 H), 3.95-4.03 (m, 1 H), 3.96 (d, 1 H), 4.62 (d, 1 H), 4.93 (d, 1 H), 5.62-5.64 (m, 1 H), 5.76-5.79 (m, 1 H), 7.16 (d, 1 H), 8.44 (d, 1 H), 8.66 (s, 1 H). logP (pH 2.7): 1.96. |
| VII-4 | COMe | COMe | 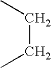 | O | 1.81-1.87 (m, 2 H), 2.05-2.08 (m, 2 H), 2.22-2.26 (m, 2 H), 2.42 (m, 2 H), 3.44 (m, 2 H), 3.75 (s, 3 H), 3.79 (s, 3 H), 4.57 (br s, 2 H), 5.55- 5.62 (m, 1 H), 5.66-5.73 (m, 1 H), 6.90 (s, 1 H), 7.09 (s, 1 H). logP (pH 2.7): 2.78. |

| | | | | |
|---|---|---|---|---|
| VII-5 | C—OCH₂O—C | 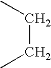 | O | 1.82-1.90 (m, 2 H), 2.02-2.07 (m, 2 H), 2.25-2.30 (m, 2 H), 2.30-2.50 (m, 2 H), 3.41 (br s, 2 H), 4.40-4.60 (br s, 2 H), 5.56-5.63 (m, 1 H), 5.71-5.78 (m, 1 H), 5.97 (s, 2 H), 6.76 (s, 1 H), 7.07 (s, 1 H). logP (pH 2.7): 2.94. |
| VII-6* | C—OCH₂O—C | 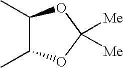 | O | 1.40 (s, 3 H), 1.45 (s, 3 H), 2.27- 2.33 (m, 1 H), 2.44-2.62 (m, 3 H), 3.26-3.33 (m, 1 H), 3.83-3.89 (m, 2 H), 4.04 (d, 1 H), 4.55 (d, 1 H), 4.83 (d, 1 H), 5.53-5.58 (m, 1 H), 5.69- 5.74 (m, 1 H), 5.95 (d, 1 H), 5.96 (d, 1 H), 6.78 (s, 1 H), 7.05 (s, 1 H). logP (pH 2.7): 2.92. |

^{a) 1}H-NMR (CD₃CN): δ [ppm]
*cf. Enders, D. et al., Angew. Chem. Int. Ed. 44, 3766-3769, 2005

### BIOLOGICAL EXAMPLES

### Example 1

Compounds of the present invention were tested in a microtubulin polymerization test from Cytoskeleton (CytoDYNAMIX Screen 03, Order No. CDS034-B, distributed by Tebu-bio GmbH, Offenbach) on purified bovine tubulin. All reagents required for tubulin polymerization, including the buffer used and the controls paclitaxel and colchicine, except for the test compounds and the solvent DMSO (dimethyl sulphoxide) were from this CytoDYNAMIX Screen 03 kit. Stock solutions of the test compounds (10 mM) were prepared in DMSO and stored at −20° C.

The tests were carried out according to the instructions of the manufacturer. Initially, the tubulin polymerization buffer (TPB) was prepared and stored at 4° C.; it comprised 910 μl of general tubulin buffer (BST01-001), 80 μl of the 60% strength glycerol buffer (BST05-001) and 10 μl of a GTP solution (BST06-001). The comparative controls also examined in the test were firstly paclitaxel as enhancer of tubulin polymerization and secondly colchicine as inhibitor of tubulin polymerization, both in a final concentration of 3 μM. The test compounds were initially added as a 10-fold concentrate to the TPB buffer; the final concentration of the test compounds were in each case 10 μM. The test compounds were compared to a tubulin solution which did not contain any test compound or control compound. In all batches, the final DMSO concentration was in each case 0.5%.

From each batch, in each case 10 μl were pipetted into a cavity of a 96-well plate. The plate was then incubated at 37° C. for 10 minutes. Tubes with tubulin (Order No. TL238), in each case 1 mg, were in each case resuspended in 310 μl of TPB at 4° C. Three times, the samples were pipetted onto ice and removed again, and the tubes then remained on ice for 1 minute.

In each case 100 μl of the tubulin solution were then pipetted into each well of the plates, which had been pre-warmed at 37° C. for 10 minutes, and the measurement was then started. The progress of the polymerization of the bovine tubulin was monitored in a Spectrafluor Plus in the measuring mode absorbance at a wavelength of 340 nm; after the start of the reaction, measurements were taken for each cavity every 2 minutes over a period of 60 minutes.

It is generally possible to demonstrate the action of compounds of the formula (I) on microtubuli in the manner described above.

### Example 2

The compounds of the general formula (I) according to the invention were tested for a possible cytotoxic or proliferation-inhibiting activity on the human tumour cell lines HeLa, SW620 and A375 (all from ATTC, American Type Culture Collection). To this end, the cells were plated in microtitre plates from Greiner (Manufacturer No. 781092) at a cell density of 1000 cells/microtitre plate well and cultivated in cell culture medium at 37° C. under a 5% carbon dioxide atmosphere. Cell culture media and additives were purchased from Invitrogen and the foetal calf serum from Biochrom. The cell culture media for Hela and A375 cells were used as stated by ATCC (HeLa: MEM, Order No. 10370-047, with 1% sodium bicarbonate, 1% non-essential amino acids, 1% sodium pyruvate, 10% foetal calf serum, 0.1% gentamycin; A375: DMEM, Order No. 41965-039, 2% sodium bicarbonate, 1% L-Glutamax, 10% foetal calf serum, 0.1% gentamycin). The culture medium for the SW620 cells consisted of DMEM, Order No. 41965-039, 1% non-essential amino acids, 10% foetal calf serum, 0.1% gentamycin.

24 hours after the cells had been plated in the microtitre plates, various concentrations of at most 100 μM down to a minimum concentration of 5 nM of the test compounds were added to the cells. Stock solutions of the test compounds (10 mM) were prepared in DMSO and stored at −20° C.; for the cytotoxicity tests, the test compounds were diluted in the appropriate cell culture medium.

After a further 48 hours of incubation, the cells were washed with medium and analysed with the aid of a two-colour fluorescence cytotoxicity/viability test (LIVE/DEAD viability/cytotoxicity ASSAY kit from Molecular Probes, Order No. L-3224), according to the instructions of the manufacturer. To this end, the medium was aspirated and in each case 30 μl of LIVE/DEAD reagent per microtitre plate well were added to the cells, which were then incubated for 30 minutes. The cells were then washed with PBS (phosphate-buffered saline). The number of live cells was analysed by measuring the green fluorescence of the live dye calcein-AM as a component of the LIVE/DEAD reagent using a fluorescence plate reader (Flexstation, from Molecular Devices) at an excitation wavelength of 485 nM and an emission wavelength of 525 nM (cf. Oral, H. B. Endothelium 6, (1998), 143-151).

Cells only with cell culture medium, without added test compounds, were treated in parallel and analysed as growth controls. The reference compounds used for anti-tumour agents having a cytotoxic or proliferation-inhibiting action were colchicine (from Merck/Calbiochem, Order No. 234115) and Taxol (baccatin III N-benzyl-b-phenylisoserine ester, from Merck/Calbiochem, Order No. 580555) (cf. Schiff, P. B. and Horwitz, S. B., Proceedings of the National Academy of Sciences of the U.S.A. 77, 1561-1565, 1980; Holmes, F. A. et al., Journal of the National Cancer Institute 83, 1797-1805, 1991).

The cytotoxic effects of the test compounds were expressed as logarithmic GI₅₀ values (growth-inhibiting logarithmic concentration value at which a cell growth reduced by 50% compared to the control without test compounds was measured (cf. Xia et al., Journal of Medicinal Chemistry 44, 3932-3936, 2001; Smith, J. A. et al., Gynecologic Oncology 98, 141-145, 2005)).

TABLE 4
Inhibition of the cell proliferation in the presence of test
compounds according to the protocol of the LIVE/DEAD
cytotoxicity assay on various human tumour cell lines,
log GI₅₀ values (M):

| Compound | A375 cells | SW620 cells | HeLa cells |
|---|---|---|---|
| Colchicine | 7.7 | 7.9 | 7.8 |
| Taxol | 6.9 | 7.0 | 7.0 |
| Cripowellin II | 8.0 | 6.9 | 8.7 |
| (Cripowellin B) | | | |

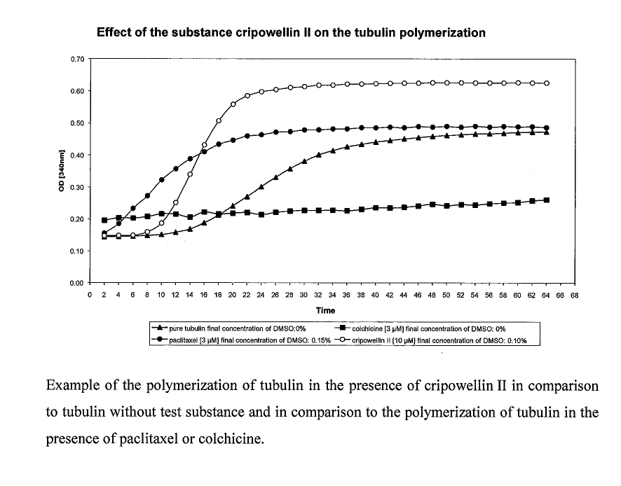

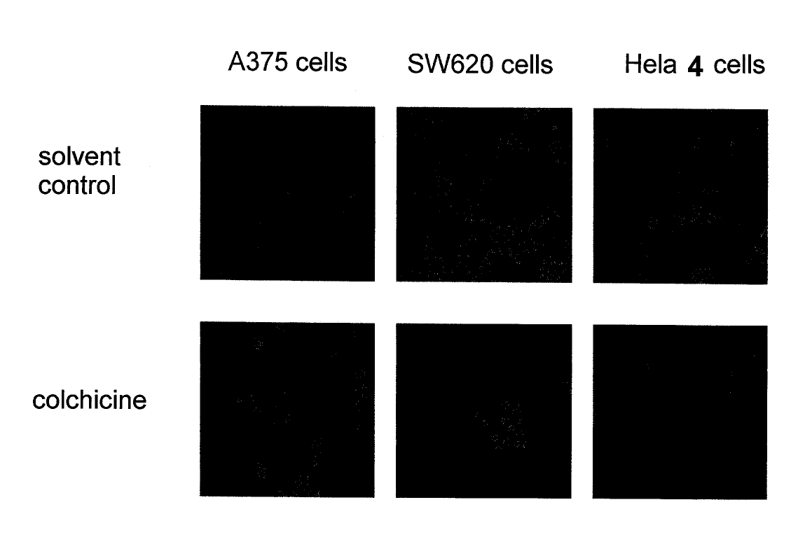

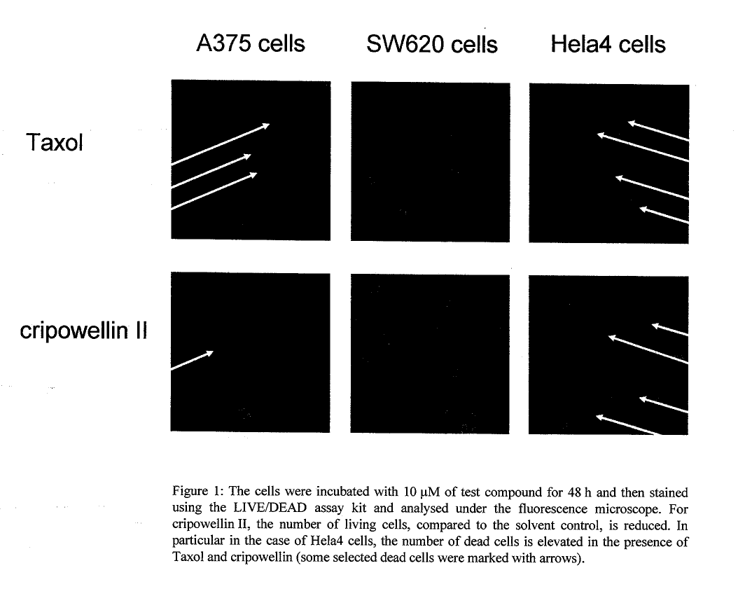

Alternatively to the cell growth analysis in microtitre plates, the different cells were also cultivated on slides and incubated analogously with the test compounds at 10 μM for a duration of 48 hours. As described, the cells were treated with the reagents from the LIVE/DEAD Assay kit according to the instructions of the manufacturer and then studied using a fluorescence microscope (FIG. 2). The live cells (measurement of the green fluorescence of the live dye calcein-AM) were analysed using an Axiovert-100 microscope (from Zeiss) at an excitation wavelength of 488 nM and an emission wavelength of 510 nM, the dead cells (measurement of the red fluorescence of the dye ethidium homodimer) were analysed at an excitation wavelength of 543 nM and an emission wavelength of 570 nM.

1. A compound of the formula (I) 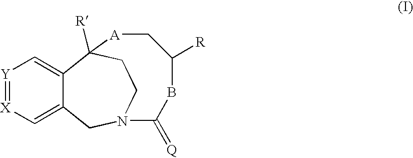
in which
R represents hydrogen or the radical —OR¹,
R′ represents hydrogen or hydroxyl,
A represents methylene, carbonyl, thiocarbonyl or the group —CH(OR²),
B represents methylene, carbonyl, thiocarbonyl or the group —CH(OR³),
Q represents oxygen or sulphur,
R¹ represents hydrogen, 2-tetrahydropyranyl, an optionally substituted glycosyl radical or one of the radicals —SO₂R⁴⁻¹, —COR⁴⁻¹, —CO₂R⁴⁻¹, —CONHR⁴⁻¹ or —CONR⁴⁻¹R⁵⁻¹,
R² represents hydrogen, 2-tetrahydropyranyl, an optionally substituted glycosyl radical or one of the radicals —SO₂R⁴⁻², —COR⁴⁻², —CO₂R⁴⁻², —CONHR⁴⁻² or —CONR⁴⁻²R⁵⁻² and
R³ represents hydrogen, 2-tetrahydropyranyl, an optionally substituted glycosyl radical or one of the radicals —SO₂R⁴⁻³, —COR⁴⁻³, —CO₂R⁴⁻³, —CONHR⁴⁻³ or —CONR⁴⁻³R⁵⁻³
in which
R⁴⁻¹, R⁴⁻², R⁴⁻³, R⁵⁻¹, R⁵⁻² and R⁵⁻³ independently of one another represent optionally halogen-substituted alkyl or optionally substituted aryl
or
R¹ and R³ together represent carbonyl, thiocarbonyl or optionally methyl-substituted alkylene,
Y═X represents a group 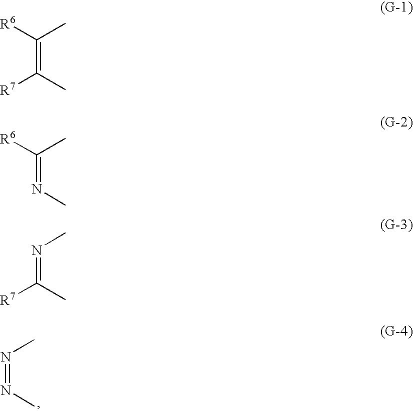
in which
R⁶ and R⁷ independently of one another represent hydrogen, halogen, hydroxyl, nitro, cyano, NR⁸R⁹, SO₂OH, SO₂NR⁸R⁹, formyl, COOH, CONR⁸R⁹, C₁₋₄-alkyl, halo-C₁₋₄-alkyl, C₁₋₄-alkylcarbonyl, halo-C₁₋₄-alkylcarbonyl, C₁₋₄-alkoxy, halo-C₁₋₄-alkoxy, C₁₋₄-alkoxycarbonyl, C₁₋₄-alkylthio, halo-C₁₋₄-alkylthio, C₁₋₄-alkylsulphinyl, halo-C₁₋₄-alkylsulphinyl, C₁₋₄-alkylsulphonyl, halo-C₁₋₄-alkylsulphonyl, C₁₋₄-alkyloxysulphonyl, C₃₋₇-cycloalkyl, optionally substituted aryl, optionally substituted aryl-C₁₋₄-alkyl, optionally substituted aryloxy, optionally substituted aryl-C₁₋₄-alkyloxy, optionally substituted hetaryl, optionally substituted hetaryloxy, optionally substituted hetaryl-C₁₋₄-alkyl or optionally substituted hetaryl-C₁₋₄-alkyloxy,
or
R⁶ and R⁷ represent a group —O—CH₂—O—, —O—CHF—O—, —O—CF₂—O—, —O—CH₂—CH₂—O—, —O—CF₂—CF₂—O—,
R⁸ and R⁹ independently of one another represent hydrogen, C₁₋₄-alkyl, halo-C₁₋₄-alkyl, C₁₋₄-alkylcarbonyl, halo-C₁₋₄-alkylcarbonyl, C₁₋₄-alkoxy, C₁₋₄-alkoxycarbonyl, C₁₋₄-alkylsulphinyl, C₁₋₄-alkylsulphonyl, C₃₋₇-cycloalkyl, C₃₋₇-cycloalkylcarbonyl, C₃₋₇-cycloalkyl-C₁₋₂-alkyl or phenyl-C₁₋₂-alkyl,
or
R⁸ and R⁹ as a group together with the linking nitrogen atoms form an optionally substituted five-, six- or seven-membered ring which is optionally interrupted by one or more heteroatoms, and 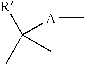
may also represent the group 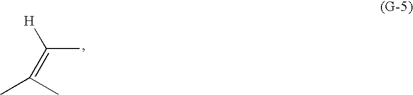
for treating a diseases of man.

2. A compound according to claim 1, wherein
R represents the radical —OR¹,
R′ represents hydrogen,
A represents carbonyl,
B represents the group —CH(OR³)—,
Q represents oxygen,
R¹ represents hydrogen, 2-tetrahydropyranyl, an optionally substituted monosaccharide radical or one of the radicals —SO₂R⁴⁻¹, —COR⁴⁻¹ or —CONHR⁴⁻¹,
R² represents hydrogen, 2-tetrahydropyranyl, an optionally substituted monosaccharide radical or one of the radicals —COR⁴⁻² or —CONHR⁴⁻²,
R³ represents hydrogen, 2-tetrahydropyranyl, an optionally substituted monosaccharide radical or one of the radicals —COR⁴⁻³ or —CONHR⁴⁻³,
R⁴⁻¹, R⁴⁻², R⁴⁻³, R⁵⁻¹, R⁵⁻² and R⁵⁻³ independently of one another represent optionally fluorine- or chlorine-substituted C₁₋₈-alkyl or optionally fluorine-, chlorine-, nitro-, C₁₋₄-alkyl- or C₁₋₄-alkoxy-substituted phenyl
or
R¹ and R³ together represent carbonyl, thiocarbonyl or optionally methyl-substituted C₁₋₃-alkylene,
Y═X represents the group 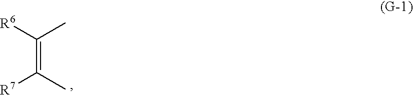
R⁶ and R⁷ independently of one another represent hydrogen, fluorine, chlorine, bromine, iodine, hydroxyl, nitro, cyano, amino, N-methylamino, N,N-dimethylamino, methyl, ethyl, n-propyl, isopropyl, n-butyl, sec-butyl, isobutyl, tert-butyl, fluoromethyl, difluoromethyl, trifluoromethyl, chlorodifluoromethyl, pentafluoroethyl, n-heptafluoropropyl, isoheptafluoropropyl, isohexafluoropropyl, methylcarbonyl, ethylcarbonyl, n-propylcarbonyl, isopropylcarbonyl, n-butylcarbonyl, sec-butylcarbonyl, isobutylcarbonyl, tert-butylcarbonyl, trifluoromethylcarbonyl, pentafluoroethylcarbonyl, methoxy, ethoxy, n-propoxy, isopropoxy, n-butoxy, sec-butoxy, isobutoxy, tert-butoxy, trifluoromethoxy, chlorodifluoromethoxy, pentafluoroethoxy, methoxycarbonyl, ethoxycarbonyl, n-propoxycarbonyl, isopropoxycarbonyl, n-butoxycarbonyl, sec-butoxycarbonyl, isobutoxycarbonyl, tert-butoxycarbonyl, methylthio, ethylthio, n-propylthio, isopropylthio, n-butylthio, sec-butylthio, isobutylthio, tert-butylthio, trifluoromethylthio, trifluoromethylsulphinyl, trifluoromethylsulphonyl, cyclopropyl, cyclobutyl, cyclopentyl, cyclohexyl, optionally substituted phenyl, optionally substituted phenyl-C₁₋₄-alkyl, optionally substituted phenoxy, optionally substituted phenyl-C₁₋₄-alkyloxy, in particular phenylmethoxy or phenylethoxy, optionally substituted pyridyl, pyrimidyl, pyrazinyl, pyrazolyl, thiazolyl, thienyl or furyl, optionally substituted pyridyloxy, pyrimidyloxy or pyrazinyloxy, optionally substituted pyridylmethyl, pyridylethyl, pyrimidylmethyl, pyrazinylmethyl or thiazolylmethyl, optionally substituted pyridylmethoxy, pyrimidylmethoxy or pyrazinylmethoxy, which may optionally be substituted by radicals from the group consisting of fluorine, chlorine, bromine and iodine, C₁₋₄-alkyl, C₃₋₆-cycloalkyl, C₃₋₆-cycloalkoxy, C₃₋₆-cycloalkyl-C₁₋₂-alkoxy, C₁₋₄-haloalkyl, amino, hydroxyl, nitro, cyano, SO₂OH, COOH, formyl, C₁₋₄-alkoxy, C₁₋₂-alkylenedioxy, C₁₋₄-haloalkoxy, C₁₋₄-alkylthio, C₁₋₄-alkylsulphinyl, C₁₋₄-alkylsulphonyl, C₁₋₄-haloalkylthio, C₁₋₄-haloalkylsulphoxyl, C₁₋₄-haloalkylsulphonyl, C₁₋₄-alkylamino, di-(C₁₋₄-alkyl)amino, C₁₋₄-alkylcarbonyl, C₃₋₆-cycloalkylcarbonyl, phenylcarbonyl, C₁₋₄-alkoxycarbonyl,
or
represent a group —O—CH₂—O—, —O—CHF—O—, —O—CF₂—O—, —O—CH₂—CH₂—O—, —O—CF₂—CF₂—O—,
R⁸ and R⁹ independently of one another represent hydrogen, methyl, ethyl, n-propyl, isopropyl, n-butyl, isobutyl, sec-butyl or tert-butyl, trifluoromethyl, difluoromethyl, monofluoropropyl, methylcarbonyl, ethylcarbonyl, trifluoromethylcarbonyl, methoxycarbonyl, ethoxycarbonyl, n-propoxycarbonyl, methylsulphinyl, ethylsulphinyl, methylsulphonyl, ethylsulphonyl, cyclopropyl, cyclopropylmethyl, cyclopropylcarbonyl, benzyl or phenethyl,
R⁸ and R⁹ as a group together with the linking nitrogen atoms represent N-pyrrolidino, N-morpholino, N-thiomorpholino, N—(N′-methyl)piperidino or a radical selected from the group consisting of (G-6) to (G-13) 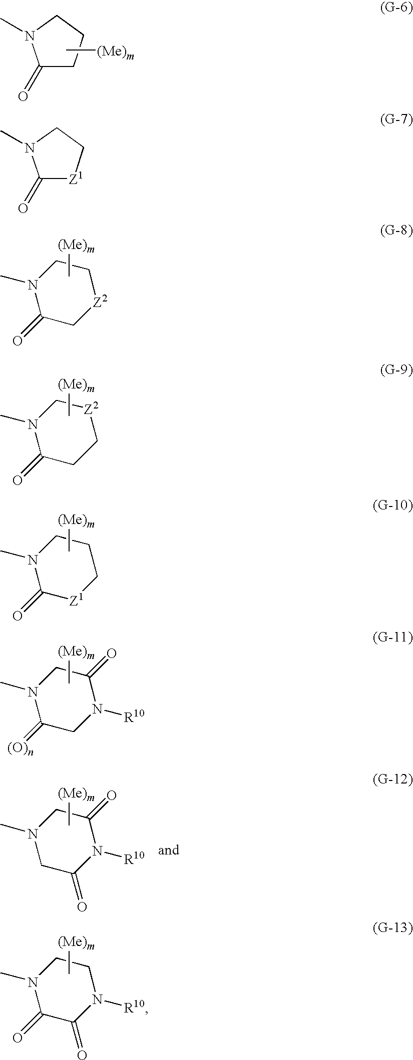
in which
Z¹ represents oxygen, methylene or N—R¹⁰,
Z² represents oxygen, sulphur, sulphinyl, sulphonyl, methylene or N—R¹⁰,
R¹⁰ represents hydrogen or C₁₋₄-alkyl, in particular methyl,
n and m independently of one another represent 0 or 1,
for treating a disease.

3. A human medicament, comprising at least one compound of formula (I) as defined in claim 1 and at least one pharmaceutically acceptable carrier or diluent.

4. A method for using a compound of formula (I) as defined in claim 1 for preparing a medicament for the treatment of cancer or other proliferative disease in man or animal.

5. A compound of formula (I) 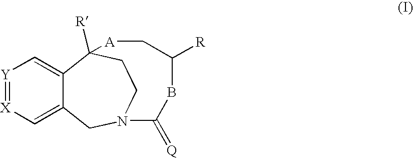
in which
R represents hydrogen or the radical —OR¹,
R′ represents hydrogen or hydroxyl,
A represents methylene, carbonyl, thiocarbonyl or the group —CH(OR²),
B represents methylene, carbonyl, thiocarbonyl or the group —CH(OR³),
Q represents oxygen or sulphur,
R¹ represents hydrogen, 2-tetrahydropyranyl, an optionally substituted glycosyl radical or one of the radicals —SO₂R⁴⁻¹, —COR⁴⁻¹, —CO₂R⁴⁻¹, —CONHR⁴⁻¹ or —CONR⁴⁻¹R⁵⁻¹,
R² represents hydrogen, 2-tetrahydropyranyl, an optionally substituted glycosyl radical or one of the radicals —SO₂R⁴⁻², —COR⁴⁻², —CO₂R⁴⁻², —CONHR⁴⁻² or —CONR⁴⁻²R⁵⁻² and
R³ represents hydrogen, 2-tetrahydropyranyl, an optionally substituted glycosyl radical or one of the radicals —SO₂R⁴⁻³, —COR⁴⁻³, —CO₂R⁴⁻³, —CONHR⁴⁻³ or —CONR⁴⁻³R⁵⁻³
in which
R⁴⁻¹, R⁴⁻², R⁴⁻³, R⁵⁻¹, R⁵⁻² and R⁵⁻³ independently of one another represent optionally halogen-substituted alkyl or optionally substituted aryl
or
R¹ and R³ together represent carbonyl, thiocarbonyl or optionally methyl-substituted alkylene,
Y═X represents a group 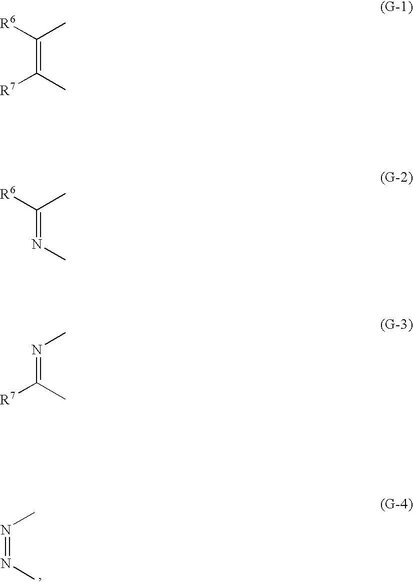
in which
R⁶ and R⁷ independently of one another represent hydrogen, halogen, hydroxyl, nitro, cyano, NR⁸R⁹, SO₂OH, SO₂NR⁸R⁹, formyl, COOH, CONR⁸R⁹, C₁₋₄-alkyl, halo-C₁₋₄-alkyl, C₁₋₄-alkylcarbonyl, halo-C₁₋₄-alkylcarbonyl, C₁₋₄-alkoxy, halo-C₁₋₄-alkoxy, C₁₋₄-alkoxycarbonyl, C₁₋₄-alkylthio, halo-C₁₋₄-alkylthio, C₁₋₄-alkylsulphinyl, halo-C₁₋₄-alkylsulphinyl, C₁₋₄-alkylsulphonyl, halo-C₁₋₄-alkylsulphonyl, C₁₋₄-alkyloxysulphonyl, C₃₋₇-cycloalkyl, optionally substituted aryl, optionally substituted aryl-C₁₋₄-alkyl, optionally substituted aryloxy, optionally substituted aryl-C₁₋₄-alkyloxy, optionally substituted hetaryl, optionally substituted hetaryloxy, optionally substituted hetaryl-C₁₋₄-alkyl or optionally substituted hetaryl-C₁₋₄-alkyloxy,
or
R⁶ and R⁷ represent a group —O—CHF—O—, —O—CF₂—O—, —O—CH₂—CH₂—O—, —O—CF₂—CF₂—O—,
R⁸ and R⁹ independently of one another represent hydrogen, C₁₋₄-alkyl, halo-C₁₋₄-alkyl, C₁₋₄-alkylcarbonyl, halo-C₁₋₄-alkylcarbonyl, C₁₋₄-alkoxy, C₁₋₄-alkoxycarbonyl, C₁₋₄-alkylsulphinyl, C₁₋₄-alkylsulphonyl, C₃₋₇-cycloalkyl, C₃₋₇-cycloalkylcarbonyl, C₃₋₇-cycloalkyl-C₁₋₂-alkyl or phenyl-C₁₋₂-alkyl,
or
R⁸ and R⁹ as a group together with the linking nitrogen atoms form an optionally substituted five-, six- or seven-membered ring which is optionally interrupted by one or more heteroatoms, and 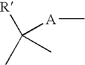
may also represent the group

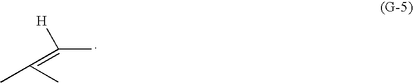

6. A compound according to claim 5, wherein
R represents the radical —OR¹,
R′ represents hydrogen,
A represents carbonyl,
B represents the group —CH(OR³)—,
Q represents oxygen,
R¹ represents hydrogen, 2-tetrahydropyranyl, an optionally substituted monosaccharide radical of one of the radicals —SO₂R⁴⁻¹, —COR⁴⁻¹ or —CONHR⁴⁻¹,
R² represents hydrogen, 2-tetrahydropyranyl, an optionally substituted monosaccharide radical or one of the radicals —COR⁴⁻² or —CONHR⁴⁻²,
R³ represents hydrogen, 2-tetrahydropyranyl, an optionally substituted monosaccharide radical or one of the radicals —COR⁴⁻³ or —CONHR⁴⁻³,
R⁴⁻¹, R⁴⁻², R⁴⁻³, R⁵⁻¹, R⁵⁻² and R⁵⁻³ independently of one another represent optionally fluorine- or chlorine-substituted C₁₋₈-alkyl or optionally fluorine-, chlorine-, nitro-, C₁₋₄-alkyl- or C₁₋₄-alkoxy-substituted phenyl
R¹ and R³ together represent carbonyl, thiocarbonyl or optionally methyl-substituted C₁₋₃-alkylene,
Y═X represents the group 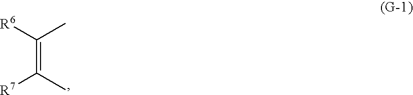
R⁶ and R⁷ independently of one another represent hydrogen, fluorine, chlorine, bromine, iodine, hydroxyl, nitro, cyano, amino, N-methylamino, N,N-dimethylamino, methyl, ethyl n-propyl, isopropyl, n-butyl, sec-butyl, isobutyl, tert-butyl, fluoromethyl, difluoromethyl, trifluoromethyl, chlorodifluoromethyl, pentafluoroethyl, n-heptafluoropropyl, isoheptafluoropropyl, isohexafluoropropyl, methylcarbonyl, ethylcarbonyl, n-propylcarbonyl, isopropylcarbonyl, n-butylcarbonyl, sec-butylcarbonyl, isobutylcarbonyl, tert-butylcarbonyl, trifluoromethylcarbonyl, pentafluoroethylcarbonyl, methoxy, ethoxy, n-propoxy, isopropoxy, n-butoxy, sec-butoxy, isobutoxy, tert-butoxy, trifluoromethoxy, chlorodifluoromethoxy, pentafluoroethoxy, methoxycarbonyl, ethoxycarbonyl, n-propoxycarbonyl, isopropoxycarbonyl, n-butoxycarbonyl, sec-butoxycarbonyl, isobutoxycarbonyl, tert-butoxycarbonyl, methylthio, ethylthio, n-propylthio, isopropylthio, n-butylthio, sec-butylthio, isobutylthio, tert-butylthio, trifluoromethylthio, trifluoromethylsulphinyl, trifluoromethylsulphonyl, cyclopropyl, cyclobutyl, cyclopentyl, cyclohexyl, optionally substituted phenyl, optionally substituted phenyl-C₁₋₄-alkyl, optionally substituted phenoxy, optionally substituted phenyl-C₁₋₄-alkyloxy, optionally substituted pyridyl, pyrimidyl, pyrazinyl, pyrazolyl, thiazolyl, thienyl or furyl, optionally substituted pyridyloxy, pyrimidyloxy or pyrazinyloxy, optionally substituted pyridylmethyl, pyridylethyl, pyrimidylmethyl, pyrazinylmethyl or thiazolylmethyl, optionally substituted pyridylmethoxy, pyrimidylmethoxy or pyrazinylmethoxy, which may optionally be substituted by radicals from the group consisting of fluorine, chlorine, bromine and iodine, C₁₋₄-alkyl, C₃₋₆-cycloalkyl, C₃₋₆-cycloalkoxy, C₃₋₆-cycloalkyl-C₁₋₂-alkoxy, C₁₋₄-haloalkyl, amino, hydroxyl, nitro, cyano, SO₂₀H, COOH, formyl, C₁₋₄-alkoxy, C₁₋₂-alkylenedioxy, C₁₋₄-haloalkoxy, C₁₋₄-alkylthio, C₁₋₄-alkylsulphinyl, C₁₋₄-alkylsulphonyl, C₁₋₄-haloalkylthio, C₁₋₄-haloalkylsulphoxyl, C₁₋₄-haloalkylsulphonyl, C₁₋₄-alkylamino, di-(C₁₋₄-alkyl)amino, C₁₋₄-alkylcarbonyl, C₃₋₆-cycloalkylcarbonyl, phenylcarbonyl, C₁₋₄-alkoxycarbonyl,
or
represent a group —O—CHF—O—, —O—CF₂—O—, —O—CH₂—CH₂—O—, —O—CF₂—CF₂—O—,
R⁸ and R⁹ independently of one another represent hydrogen, methyl, ethyl, n-propyl, isopropyl, n-butyl, isobutyl, sec-butyl or tert-butyl, trifluoromethyl, difluoromethyl, monofluoropropyl, methylcarbonyl, ethylcarbonyl, trifluoromethylcarbonyl, methoxycarbonyl, ethoxycarbonyl, n-propoxycarbonyl, methylsulphinyl, ethylsulphinyl, methylsulphonyl, ethylsulphonyl, cyclopropyl, cyclopropylmethyl, cyclopropylcarbonyl, benzyl or phenethyl,
R⁸ and R⁹ as a group together with the linking nitrogen atoms represent N-pyrrolidino, N-morpholino, N-thiomorpholino, N—(N′-methyl)piperidino or a radical selected from the group consisting of (G-6) to (G-13) 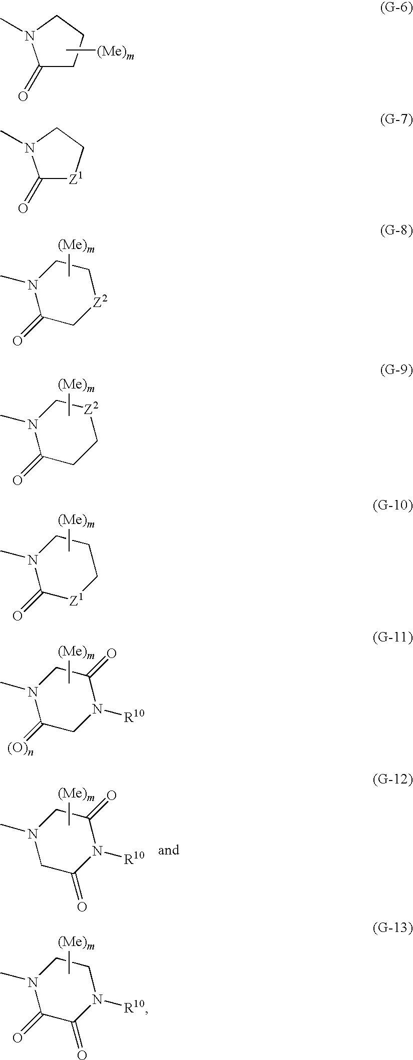
in which
Z¹ represents oxygen, methylene or N—R¹⁰,
Z² represents oxygen, sulphur, sulphinyl, sulphonyl, methylene or N—R¹⁰,
R¹⁰ represents hydrogen or C₁₋₄-alkyl, in particular methyl, and
n and m independently of one another represent 0 or 1.

7. A process for preparing a compound according to claim 5, formula (I) 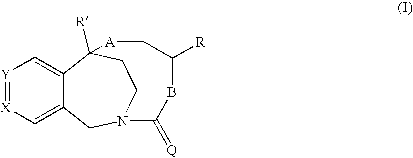
said process comprising:
a) reacting compounds of formula (II) 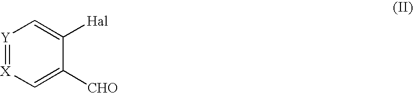
in which
Hal represents a halogen,
in a first reaction with 3-buten-1-amine, if appropriate in the presence of a diluent and if appropriate in the presence of an acidic reaction auxiliary and if appropriate with removal of water, to give a compound the of formula (III) 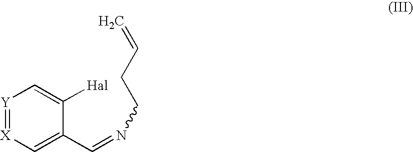
then, conducting a conversion in a second reaction step, if appropriate in situ and if appropriate in the presence of a diluent and if appropriate in the presence of a hydrogenating agent, into a compound of formula (IV) 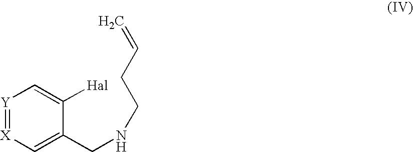
then, reacting in a third reaction step, if appropriate in the presence of a diluent and if appropriate in the presence of a basic reaction auxiliary, with a compound of formula (V) 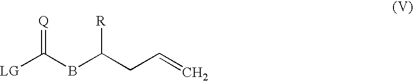
in which
LG is a nucleofugic leaving group, if appropriate generated in situ, and
if appropriate in the presence of a diluent and if appropriate in the presence of a basic reaction auxiliary, to give a compound of formula (VI), 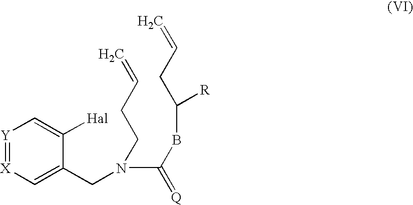
and conducting a conversion in a fourth reaction step, under reaction conditions of a metathesis reaction, if appropriate in the presence of a suitable catalyst and if appropriate in the presence of a diluent, into a compound of formula (VII) 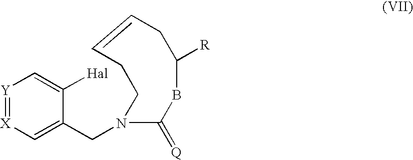
and, in a fifth reaction step, conducting a conversion under reaction conditions of a Heck reaction, if appropriate in the presence of a suitable noble metal salt and if appropriate in the presence of a suitable catalyst and if appropriate in the presence of a diluent, into a compound of formula (Ia),
and/or
b) a compound of formula (Ia) 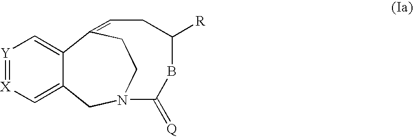
is converted by oxidation of the C═C double bond, if appropriate in the presence of a diluent, into a compound of formula (I) 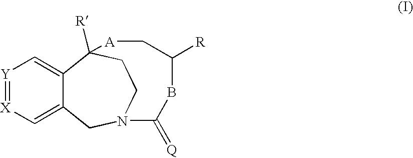
in which
R′ represents hydroxyl, and
A represents CH—OH,
and then, in a second reaction step, by oxidation of the CH—OH group A in the presence of a diluent, conducting a conversion into a compound of formula (I)
in which
R′ represents hydroxyl,
and in a third reaction step, in the presence of a suitable salt of a lanthanoid metal, if appropriate in the presence of a diluent, conducting a conversion into a compound of formula (I)
in which
R′ represents hydrogen.
A, B, R, Q, X and Y are as defined above.

8. A process according to claim 7, wherein Hal represents bromine.

9. A process according to claim 7 wherein Q represents oxygen.
